SEQUENCE LISTING (1 sequence)

SEQ ID NO: 1 - protein, 29 aa, synthetic construct
  LIPID 24: note = The epsilon amino group of Lys is substituted with([17-carboxy-heptadecanoyl]-isoGlu-GSGSGG)-
  REGION 1..29: note = glucagon analogue
  SITE 2: note = 1-amino-cyclobutanecarboxylic acid
HXQGTFTSDYSKYLDERAAKDFIKWLESA

(19) United States
(12) Patent Application Publication
(10) Pub. No.: US 2025/0262279 A1
(43) Pub. Date: Aug. 21, 2025

(54) DOSE REGIMEN FOR LONG-ACTING GLP1/GLUCAGON RECEPTOR AGONISTS

(71) Applicant: Boehringer Ingelheim International GmbH, Ingelheim am Rhein (DE)

(72) Inventors: Michael DESCH, Kirchham (DE); Anita Magdalena HENNIGE, Biberach an der Riss (DE); Corinna Isabel SCHOELCH, Mittelbiberach (DE); Claus THAMER, Ulm (DE); Jan Per Martin BERGSTRAND, Uppsala (SE)

(21) Appl. No.: 18/291,966

(22) PCT Filed: Jul. 28, 2022
(86) PCT No.: PCT/EP2022/071281

§ 371 Date: Jan. 25, 2024

(30) Foreign Application Priority Data
Jul. 30, 2021 (EP) 21188741.9

(51) Int. Cl. A61K 38/26; A61K 9/00; A61P 3/04

(52) U.S. Cl. CPC A61K 38/26; A61K 9/0019; A61P 3/04

(57) ABSTRACT

The present invention relates to a dosing scheme for long-acting GLP1/glucagon receptor agonists. According to the dosing scheme the interval between two consecutive administrations is defined such that the ratio between the plasma half-life in humans of the agonist and the administration interval is more than 1.

### RELATED APPLICATION DISCLOSURE

This application is a 35 U.S.C. § 371 application from PCT/EP2022/071281, filed 28 Jul. 2022, pending, which claims the benefit of EP Provisional Application No. EP 21 188 741.9, filed Jul. 30, 2021, now expired, each of which is hereby incorporated by reference herein in its entirety.

### SEQUENCE DISCLOSURE

This application includes, as part of its disclosure, a “Sequence Listing XML” pursuant to 37 C.F.R. § 1.831(a) which is submitted in XML file format via the USPTO patent electronic filing system in a file named “01-3511-US-1_SL-2024-01-23.xml”, created on Jan. 23, 2024, and having a size of 4 kilobytes, which is hereby incorporated by reference herein in its entirety.

### FIELD OF THE INVENTION

The present invention relates to the medical use of a long-acting glucagon analogue having GLP1/glucagon receptor agonist activity. A dose regimen is disclosed showing improved tolerability.

### BACKGROUND OF THE INVENTION

Glucagon-like-protein 1 (GLP1) receptor agonists recently became a treatment option not only for type 2 diabetes mellitus but also obesity. Currently, several compounds which activate both the GLP1 receptor and the glucagon receptor are in clinical development. GLP1 receptor (GLP1R) agonism achieves glucose lowering by inducing glucose dependent insulin-secretion acting at the pancreatic β-cell. In addition, GLP1R agonists lower body weight by the inhibition of food intake due to centrally mediated mechanisms and also by inhibition of gastric emptying and intestinal transit. Glucagon (GCG) receptor agonism reduces body weight by increasing energy expenditure and might positively affect lipid metabolism resulting in lowering of plasma and liver triglycerides and plasma cholesterol. In patients with type 2 diabetes, overweight, obesity, and NASH, dual agonism at the GLP1R and GCG receptor (GCGR) is expected to result in HbA1c reduction combined with body weight loss and improvement in NASH. Simultaneous activation of the GLP1 and glucagon receptor of dual GLP1R/GCGR agonists is expected to result in a longer lasting negative energy balance than with pure GLP1 receptor agonists and to lead to robust weight loss and improvement in NASH. The balance of GLP1 and glucagon receptor activation is hypothesized to be the key factor for achieving weight loss and maintenance in the presence of a favorable benefit-risk profile, as well as improving NASH.

Until now, a few clinical dual GLP1/GCGR agonists advanced into Phase 2 clinical development. The most common adverse events (AE) in the clinical studies were nausea, vomiting, diarrhoea, headache, and increases in heart rate. These events seem to be dose-dependent. Gastrointestinal (GI) side effects are well known side effects for GLP1R agonists. Multiple studies and clinical experience from GLP1R agonists suggest that GI side effects can be mitigated by dose escalation. Accordingly, for a number of compounds of this class it was shown in clinical trials that dose escalation (up-titration) is able to significantly improve tolerability.

WO 2014/091316 relates to co-agonists of glucagon and GLP1. WO 2017/153575 discloses further data of these co-agonists, including data from clinical trials of G933.

WO 2014/056872 A1 and WO 2018/100174 A1 disclose exendin-4 derivatives, which activate the GLP1 and the glucagon receptor. In these exendin-4 derivatives, among other substitutions, methionine at position 14 is replaced by an amino acid carrying an NH₂ group in the side chain, which is further substituted with a non-polar residue (e.g. a fatty acid optionally combined with a linker).

### Compound I

WO2015/055801 and WO2015/055802 disclose glucagon analogue peptides having increased selectivity for the GLP1 receptor as compared to human glucagon. Tables 2 and 3 in WO2015/055801 provide EC50 values for the different analogues on endogenous GLP1 receptors (Example 3) and on the endogenous glucagon receptor (Example 4). Compound 13 has the following sequence and structure

H-H-Ac4c-QGTFTSDYSKYLDERAAKDFI-K([17-carboxy-heptadecanoyl]-isoGlu-GSGSGG)-WLESA-NH₂ (SEQ ID NO.: 1),

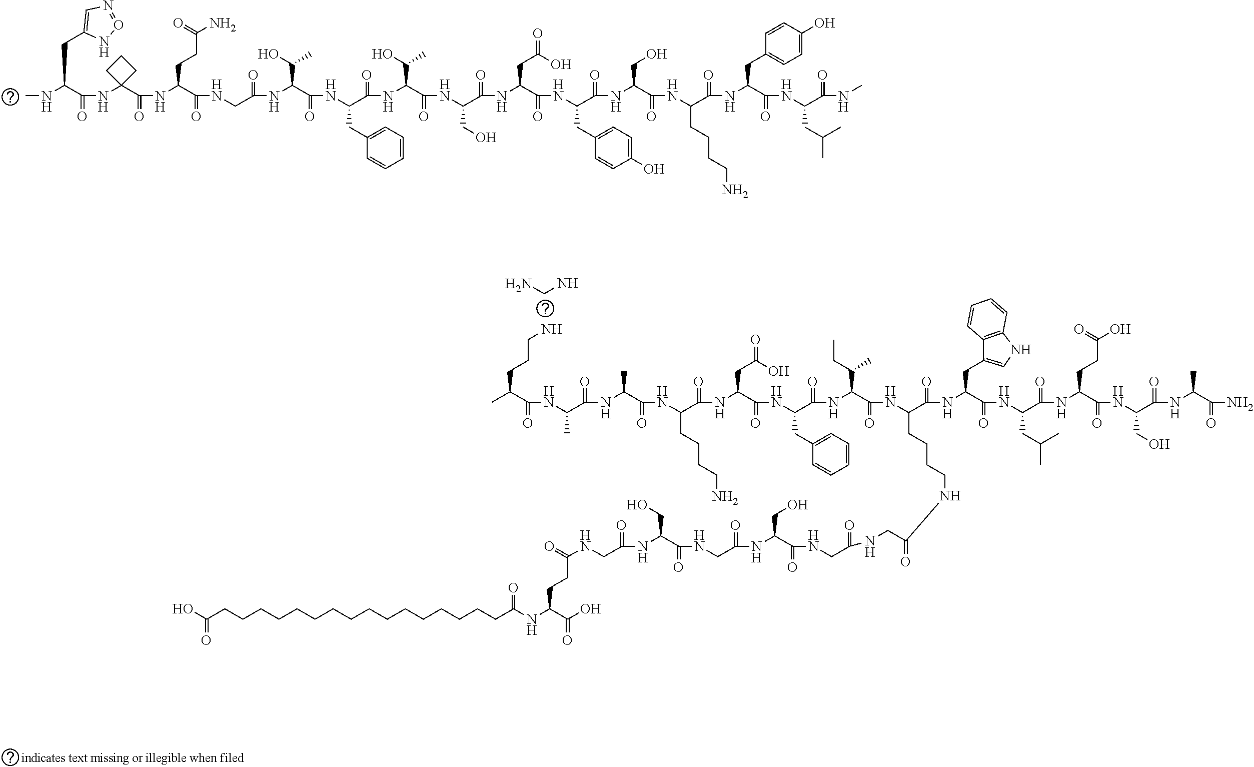

and is herein referred to as Compound I.

Compound I is a dual GLP1R and GCGR agonist as determined by their capability to stimulate intracellular cAMP formation in appropriate assays (e.g. as disclosed in WO2015/055801, Example 2, page 36, Table 1, and Examples 3 and 4, pages 37-40, Table 2 and 3).

Compound I may be manufactured in the form of an amorphous solid, for instance in the form of a sodium salt. Compound I may be formulated as an aqueous solution comprising Compound I (or its sodium salt) and other pharmaceutically acceptable excipients generally known to a skilled person, and administered via subcutaneous injection.

Compound I was recently studied in a first-in-man Phase I single-rising-dose study. In this study, a dose of 0.3 mg led to ‘mild loss of appetite’ in 33% of healthy subjects. This specific AE increased to 50% in the 0.5 mg dose group. Dose escalation to 1.2 mg was associated with 83% of the subjects experiencing moderate to severe nausea for 1 to 6 days and mild to severe vomiting.

The half-life of Compound I in humans is estimated to be around 110 h, which allows for a once weekly treatment regime.

Further methods are warranted that improves the tolerability of GLP1/GCGR dual (peptidic) agonists, such as Compound I. More specifically, new methods (such as dosage regimes or dose escalation regimes) are required that reduce undesired adverse events but at the same time allow to exploit the desired pharmacological effect (e.g. weight reduction, glucose management or reduction in liver fat).

### SUMMARY OF THE INVENTION

Provided herein is the medical use of a peptidic GLP1/glucagon receptor agonist (e.g. Compound I), wherein
a) the agonist has a plasma half-life in humans of at least 60 hours;
b) the agonist is subcutaneously administered at least two times; characterized in that
c) the interval between two consecutive administrations is such that the ratio between the plasma half-life in humans of the agonist and the administration interval is more than 1.0.

Accordingly, a dosing scheme for a peptidic GLP1/glucagon receptor agonist (e.g. Compound I) for humans is provided, wherein the agonist has a plasma half-life in humans of at least 60 hours, characterized in that the interval between two consecutive administrations of the agonist is such that the ratio between the plasma half-life in humans of the agonist and the administration interval is more than 1.0.

In some embodiments, the ratio between the plasma half-life in humans of the agonist and the administration interval is between 1.0 and 5.0.

The agonist can be administered twice per week (twice weekly, also denoted as ‘bw’ herein) or more often, e.g. every second day or daily.

The GLP1/glucagon receptor agonist is a peptide, e.g. a long-acting peptide. The peptide may bear a half-life extending moiety. In this regard, the peptide may be an acylated, long-acting glucagon or GLP1 analogue having activity at both receptors.

In a specific embodiment the agonist is Compound I. Accordingly, a dosing scheme for Compound I for humans is provided, characterized in that the interval between two consecutive subcutaneous administrations of Compound I is such that the ratio between the plasma half-life in humans of Compound I and the administration interval is more than 1.0.

Accordingly, a dosing scheme for a peptidic GLP1/glucagon receptor agonist (e.g. Compound I) for humans is provided, wherein the agonist has a plasma half-life in humans of at least 60 hours, characterized in that the interval between two consecutive administrations of the agonist is such that the ratio between the plasma half-life in humans of the agonist and the administration interval is more than 1.0.

The agonist can be used in the treatment of obesity, type II diabetes melitus (T2DM), non-alcoholic fatty liver disease (NAFLD) (including non-alcoholic steatohepatitis (NASH), non-alcoholic fatty liver (NAFL) or NAFLD-associated liver fibrosis and/or cirrhosis).

Subcutaneous injections have some drawbacks with respect to patient convenience. The patient might experience pain or discomfort during injection, or there might be some inconvenience with respect to local tolerability at the injection site. Therefore, the number of injections is generally to be kept to a minimum.

However, it now has been found that when a peptidic GLP1/glucagon agonist is administered more frequently (i.e. when the interval between two drug administrations are shorter), tolerability of the agonist can be increased (i.e. the numbers or the severity of adverse events are reduced). Therefore, in some cases a shorter administration interval (according to the dosing scheme of the present invention) might lead to an overall increase in patient convenience.

In clinical trials with Compound I it has been found that dosing schemes according to the invention induced less adverse events than conventional dosing schemes. At the same time, the desired pharmacological effect (e.g. body weight loss) obtained with the dosing scheme of the invention was not diminished but is maintained or even increased. For the sake of this comparison the total quantity administered to the patient over a certain time period is identical, but the quantity per injection is reduced and the number of injections is increased.

This is illustrated by the following observation made in a clinical trial with Compound I (a GLP1/glucagon receptor dual agonist, see Example 1):

Patients were to follow a determined dose escalation (up-titration) scheme. However, in case of side effects the patients could decide not to increase the drug load further but can stay on the current dose level. Part A of the study included one daily dosing scheme and three weekly dosing schemes. Patients in the Daily dosing group received 10.5 mg Compound I in total after treatment. No patient has withdrawn the up-titration treatment. The planned total dose of Compound I per patient based on pre-specified up-titration in the weekly dosing schemes was 6.30 mg (Weekly 1), 8.40 mg (Weekly 2), and 9.60 mg (Weekly 3). However, due to adverse events some patients stopped up-titration (i.e. they continued treatment but stayed on the same drug level) the actual mean total dose of per patient in the weekly dosing schemes was 4.75 mg (SD 2.04) for Weekly 1, 6.38 mg (SD 2.58) for Weekly 2, and 6.85 mg (SD 2.53) for Weekly 3. This means tolerability of the drug is increased with the daily dosing scheme (dosing scheme according to the invention). Although, the total amount of Compound I administered is higher, there were fewer drug-related cases in the Daily dosing group than in the other Compound I groups, e.g. for abdominal distension (Daily: 13.3%; weekly Compound I groups: from 38.9% to 52.9%) and vomiting (Daily: no cases; weekly Compound I groups: from 23.5% to 38.9%).

Further aspects and embodiments of the present invention will become apparent from the disclosure below.

### DETAILED DESCRIPTION OF THE INVENTION

Unless otherwise defined herein, scientific and technical terms used herein shall have the meanings that are commonly understood by those of ordinary skill in the art.

Throughout this specification, the word “comprise” or variations such as “comprises” or “comprising” will be understood to imply the inclusion of a stated integer or component, or of a stated group of integers or components, but not the exclusion of any other integer or component or group of integers or components.

The singular forms “a,” “an,” and “the” include the plurals unless the context clearly dictates otherwise.

The term “including” is used to mean “including but not limited to.” “Including” and “including but not limited to” are used interchangeably.

The terms “patient”, “subject,” and “individual” may be used interchangeably and refers to a human animal.

The above definition of Compound I comprises neutral and corresponding charged states of Compound I. Charged states of Compound I are for example present when the compound is in the form of a salt, e.g. a pharmaceutically acceptable salt, or in a solution, specifically in an aqueous solution.

As used herein, the term “pharmaceutically acceptable salt” is intended to indicate a salt which is not harmful to a patient or subject to which the salt in question is administered. It may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R¹)(R²)(R³)(R⁴)⁺, where R¹, R², R³ and R⁴ independently will typically designate hydrogen, optionally substituted C₁₋₆-alkyl or optionally substituted C₂₋₆-alkenyl. Examples of relevant C₁₋₆-alkyl groups include methyl, ethyl, 1-propyl and 2-propyl groups. Examples of C₂₋₆-alkenyl groups of possible relevance include ethenyl, 1-propenyl and 2-propenyl. Other examples of pharmaceutically acceptable salts are described in the “Encyclopedia of Pharmaceutical Technology”, 3^{rd} edition, James Swarbrick (Ed.), Informa Healthcare USA (Inc.), NY, USA, 2007, Vol. 5, p. 3177, and in J. Pharm. Sci. 66:1, 1-19 (1977).

The term “dose escalation” or “up-titration” refers herein to a progressive increase in the dose of a GLP1/Glucagon receptor agonist.

The term “agonist” as employed in the context of the invention refers to a substance that activates the receptor type in question, typically by binding to it (i.e. as a ligand).

The term “GLP1/glucagon receptor agonist” refers to an agonist that is able to bind to and activate the human GLP-1 and glucagon receptors.

The term “human dosing scheme” refers to a dosing scheme that is to be applied for human patients.

The term “administration interval” refers to the time span between two consecutive injections of the GLP1/Glucagon agonist.

Throughout this specification, the conventional one letter and three letter codes for naturally occurring amino acids are used, as well as generally accepted abbreviations for other amino acids, such as Ac4c (1-amino-cyclobutanecarboxylic acid). Unless otherwise indicated, reference is made to the L-isomeric forms of the amino acids in question. The term “isoGlu” refers to a γ-glutamic acid unit.

Additional abbreviations include the following:
AE: adverse event
AUC: area under the concentration-time curve of the analyte in plasma
BMI: body mass index
biw: two times per week
Cl: confidence interval
GI: gastrointestinal
MedDRA: medical dictionary for drug regulatory activities
NAFL: non-alcoholic fatty liver
NAFLD: non-alcoholic fatty liver disease
NAS: NAFLD activity score
NASH: non-alcoholic steatohepatitis
PT: preferred term
SD: standard deviation
SOC: system organ class
TS: treated set

The term “therapeutically effective amount” as used herein in the context of the herein-described methods of treatment or other therapeutic interventions according to the invention refers to an amount that is sufficient to cure, ameliorate, alleviate or partially arrest the clinical manifestations of the particular disease, disorder or condition that is the object of the treatment or other therapeutic intervention in question e.g. as measured by established clinical endpoints or other biomarkers (established or experimental), including liver biopsies. A therapeutically relevant amount may be determined empirically by one skilled in the art based on the indication being treated or prevented and the subject to whom the therapeutically relevant amount is being administered. For example, the skilled worker may measure one or more of the clinically relevant indicators of bioactivity described herein, e.g. liver fat content via MRI-PDFF, body weight or NAS (NAFLD activity score). The skilled worker may determine a clinically relevant amount through in vitro or in vivo measurements. Other exemplary measures include fibrosis markers (serum or plasma), weight loss, change in histological scores of NASH or fibrosis, liver fat content reduction, and change of liver enzymes.

An amount adequate to accomplish any or all of these effects is defined as a therapeutically effective amount. The administered amount and the method of administration can be tailored to achieve optimal efficacy. An amount effective for a given purpose will depend, inter alia, on the severity of the disease, disorder or condition that is the object of the particular treatment or other therapeutic intervention, on the body weight and general condition of the subject in question, on diet, on possible concurrent medication, and on other factors well known to those skilled in the medical arts.

Determination of an appropriate dosage size and dosing regimen most appropriate for administration of a peptide or pharmaceutically acceptable salt thereof according to the invention to a human may be guided by the results obtained by the present invention, and may be confirmed in properly designed clinical trials. An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are well known to the skilled person.

The terms “treatment” and grammatical variants thereof (e.g. “treated”, “treating”, “treat”) as employed in the present context refer to an approach for obtaining beneficial or desired clinical results. For the purposes of this invention, beneficial or desired clinical results include, but are not limited to, alleviation of symptoms, diminishment of extent of disease, stabilization (i.e. not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g. a human) in need of treatment may thus be a subject already afflicted with the disease or disorder in question. The term “treatment” includes inhibition or reduction of an increase in severity of a pathological state or symptoms (e.g. progression to cirrhosis, progression of fibrosis or worsening of NASH, e.g. increase of NAS) relative to the absence of treatment, and is not necessarily meant to imply complete cessation of the relevant disease, disorder or condition.

The Pathology Committee of the NASH Clinical Research Network (CRN) developed the so-called “NAFLD activity score (NAS)” for use in clinical trials and is described in Kleiner et al., Hepatology 2005, vol. 41, pp. 1313-1321.

In a first aspect, the invention relates to a peptidic GLP1/glucagon receptor dual agonist for use as medicament, wherein
a) the agonist has a plasma half-life in humans of at least 60 hours;
b) the agonist is subcutaneously administered at least two times; and wherein
c) the interval between two consecutive administrations is such that the ratio between the plasma half-life in humans and the administration interval of the agonist is more than 1.0.

The agonist can be administered two times per week (twice weekly, biw) or more often, e.g. every second day or daily.

The agonist may be administered over a longer period (e.g. over weeks, months or chronically) according to the above scheme. Therefore, the agonist can be administered at least 4 times, e.g. at least 6, 8 or 10 times.

The invention also relates to a dosing scheme for a peptidic GLP1/glucagon agonist (e.g. Compound I), wherein the agonist has a plasma half-life in humans of at least 60 hours, characterized in that the interval between two consecutive subcutaneous administrations of the agonist is such that the ratio between the plasma half-life in humans of the agonist and the administration interval is more than 1.0.

In a further embodiment, the GLP1/glucagon receptor agonist is a long-acting peptide.

The peptide may bear a half-life extending moiety. In this regard, the peptide may be an acylated long-acting peptide, an acylated glucagon or GLP1 analogue having activity at both receptors.

The peptide may consist of 45 amino acids or less, e.g. the peptide consists of 27 to 45 amino acids, e.g. of 29 to 39 amino acids or of 29 to 31 amino acids.

The peptide may be a GLP1 analogue with a sequence identity of 50% or higher (e.g. 60%, 70%, or 80% or higher) when compared with human GLP1(7-36).

The peptide may be a glucagon analogue with a sequence identity of 50% or higher (e.g. 60%, 70%, or 80% or higher) when compared with human glucagon. The sequence of the peptide may have a sequence identity of 60% to 85% with the sequence of human glucagon.

The peptide may be an exendin-4 analogue with a sequence identity of 50% or higher (e.g. 60%, 70%, or 80% or higher) when compared with exendin-4.

The peptide may be an oxyntomodulin analogue with a sequence identity of 50% or higher (e.g. 60%, 70%, or 80% or higher) when compared with human oxyntomodulin.

In some embodiments, Tₘₐₓ of the agonist after subcutaneous administration in humans is 48 h or shorter. In some embodiments, Tₘₐₓ is 40 h or shorter, or 30 h or shorter, respectively.

In some embodiments, the ratio between the plasma half-life in humans of the agonist and the administration interval is more than 1.1, or more than 1.4, more than 2.0, more than 3.0, or more 4.0, respectively.

In some embodiments, the ratio is between 1.0 and 5.0. In further embodiments, the ratio is between 1.5 and 5.0 or between 2.0 and 5.0.

In further embodiments, the agonist is administered several times (e.g. at least 3, 5, 10, 20, 40 times). As such, the administration can be followed for an extended period. The dosing scheme can be applied during dose escalation of the agonist, i.e. the amount of agonist administered is increased with time (e.g. with each consecutive administration, or with every second, third, fourth, etc. consecutive administration). Typically, the dose of a long acting peptidic agonist is increased after one, two, three or four weeks at a certain dose-level. The dosing scheme according to the invention might be specifically advantageous during dose escalation of a GLP1/glucagon agonist. It is known that adverse events from administration of peptidic GLP1 agonists can be reduced, or their severity can be alleviated, by gradual dose escalation of the drug level. The dosing scheme according to the present invention can further decrease adverse events and/or may allow a stringent dose escalation cascade, which allows to exploit an early onset of the beneficial pharmacological effects. Both effects lead to increased benefit for the patients.

However, the dosing scheme might also be applied during maintenance of a certain (final) drug level. As such, the dosing scheme can be applied sub-chronically or chronically.

In a further embodiment, the peptide agonist is in the form of a salt, more specifically in the form of a pharmaceutically acceptable salt.

In specific embodiment, the peptide agonist is Compound I. Accordingly, a dosing scheme for Compound I is provided, characterized in that the interval between two consecutive subcutaneous administrations of Compound I is such that the ratio between the plasma half-life in humans of Compound I and the administration interval is more than 1.0.

In an embodiment, the agonist is for use in the treatment of obesity, type II diabetes melitus (T2DM), non-alcoholic fatty liver disease (NAFLD) (including non-alcoholic steatohepatitis (NASH), non-alcoholic fatty liver (NAFL) or NAFLD-associated liver fibrosis and/or cirrhosis).

The agonists can be used for direct or indirect therapy of any condition caused or characterised by excess body weight or for treatment for chronic weight management (weight reduction & maintenance). Therefore, the agonists may be used in patients with obesity or overweight with additional co-morbidities, such as type 2 diabetes, hypertension, dyslipidemia, sleep apnea and cardiovascular disease. Further diseases that might be addressed include morbid obesity, obesity linked inflammation, obesity linked gallbladder disease, or obesity induced sleep apnea. The agonists might be used in patients having a BMI of 27 kg/m² or higher, or in patient having a BMI of 30 kg/m² or higher.

The agonists may also be used for the prevention of conditions caused or characterised by inadequate glucose control or dyslipidaemia (e.g. elevated LDL levels or reduced HDL/LDL ratio), diabetes (especially Type 2 diabetes), metabolic syndrome, hypertension, atherogenic dyslipidemia, atherosclerosis, arteriosclerosis, coronary heart disease, peripheral artery disease, stroke or microvascular disease.

Further, the agonists may be used in the treatment of NASH, optionally NASH associated with liver fibrosis (e.g. advanced liver fibrosis), NAFLD-associated liver fibrosis, e.g. advanced liver fibrosis (fibrosis stage moderate (F2) and severe (F3)). The agonist can be used for the treatment of NASH in patients having a NAS score of at least 2 (or of at least 3 or of at least 4). In more specific embodiments, at least 1 point of the NAS score arises from the ballooning subscore or, alternatively, at least 1 point of the NAS score arises each from the ballooning and the inflammation subscore.

In another aspect, the invention relates to a peptidic GLP1 and glucagon receptor agonist (e.g. Compound I) for use in a method of treating T2DM, obesity or NASH, wherein
a) the agonist has a plasma half-life in humans of at least 60 hours;
b) the agonist is subcutaneously administered at least two times;
c) the agonist is administered such that the ratio between the plasma half-life in humans of the agonist and the administration interval is more than 1.0.

In a related aspect, the invention relates to a pharmaceutical composition comprising a peptidic GLP1/Glucagon receptor agonist (e.g. Compound I) for use in a method of treating T2DM, obesity or NASH, wherein
a) the agonist has a plasma half-life in humans of at least 60 hours;
b) the agonist is subcutaneously administered at least two times;
c) the agonist is administered such that the ratio between the plasma half-life in humans of the agonist and the administration interval is more than 1.0.

All publications, patents and published patent applications referred to in this application are incorporated by reference herein, specifically the content of WO2015/055801 is incorporated by reference. In case of conflict, the present specification, including its specific definitions, will control.

Each embodiment of the invention described herein may be taken alone or in combination with one or more other embodiments of the invention.

### SHORT DESCRIPTION OF THE FIGURES

FIG. 1:

a) Patients with drug-related AEs as assessed by the investigator with an overall frequency ≥5% at the PT level (Example 1, Part A—TS)
b) Patients with drug-related AEs as assessed by the investigator with an overall frequency ≥5% at the PT level (Example 1, Part B—TS)
c) Time profile of body weight change from baseline, means and SDs per treatment group, PDS-A* (Example 1, Part A)
d) Time profile of body weight change from baseline, means and SDs per treatment group, PDS-B* (Example 1, Part B)

### *Pharmacodynamic Sets PDS-A and PDS-B:

Includes all evaluable patients from the treated set of trial Part A and B, respectively, who provided at least one evaluable observation for one of the exploratory biomarkers and without important protocol deviations relevant to the evaluation of pharmacodynamics. It was used for the pharmacodynamic (biomarker) analysis.

### EXAMPLES—CLINICAL TRIALS

### Example 1: A Study to Test Different Doses of Compound I in Patients with Obesity

A phase I, blinded within dose groups, multiple dose, placebo-controlled study to assess the safety, tolerability, pharmacokinetics and pharmacodynamics of different titration schemes of Compound I in patients with obesity and overweight

### Objectives

The main objective of the study was to investigate the safety and tolerability of different titration schemes of Compound I in otherwise healthy patients categorised as obese or overweight, and to determine an up-titration scheme that minimizes gastrointestinal adverse events (AEs).

### Methodology

Blinded within dose groups, randomised, placebo-controlled

### Number of Patients

Planned: Entered: 117
Actual: Screened: 109 (Part A) and 70 (Part B)

Number of patients Actual (cont.):

| | | |
|---|---|---|
| | | Analysed |
| Entered | Treated | (primary endpoint) |

Part A dosing schemes, 6-week up-titration¹

| | | | |
|---|---|---|---|
| Daily dosing | 16 | 15 | 15 |
| Weekly dosing 1 | 19 | 17 | 17 |
| Weekly dosing 2 | 18 | 18 | 18 |
| Weekly dosing 3 | 17 | 17 | 17 |
| Placebo | 13 | 13 | 13 |

Part B dosing schemes, 16-week up-titration¹

| | | | |
|---|---|---|---|
| Weekly dosing 4 | 13 | 13 | 13 |
| Weekly dosing 5 | 13 | 11 | 11 |
| Weekly dosing 6 | 13 | 12 | 12 |
| Placebo | 9 | 9 | 9 |

¹Details of the dosing schemes are provided below

### Diagnosis

Patients categorised as obese or overweight but otherwise healthy

### Main Criteria for Inclusion

Male patients (Part A); male and female patients (Part B)
Age 18 to <70 years
BMI 27 to <40 kg/m²
Body weight of at least 70 kg for women and 80 kg for men
Stable body weight, defined as no more than 5% change within 3 months prior to screening

Compound I solution for injection (2 mg/mL)

### Dose: Part A, 6-Week Up-Titration

Daily dosing: 0.1, 0.15, 0.2, 0.25, 0.35, 0.45 mg once daily for 1 week
Weekly dosing 1: 0.3, 0.6, 0.9, 1.2, 1.5, 1.8 mg once weekly
Weekly dosing 2: 0.4, 0.8, 1.2, 1.6, 2.0, 2.4 mg once weekly
Weekly dosing 3: 0.6, 0.6, 1.2, 1.8, 2.4, 3.0 mg once weekly

### Part B, 16-Week Up-Titration

Weekly dosing 4:
0.6, 0.6, 0.6, 0.6, 1.2, 1.2, 1.2, 1.2, 1.8, 1.8, 1.8, 1.8, 2.4, 2.4, 2.4, 2.4 mg once weekly
Weekly dosing 5:
0.6, 0.6, 1.2, 1.2, 1.8, 1.8, 2.4, 2.4, 3.0, 3.0, 3.6, 3.6, 4.2, 4.2, 4.8, 4.8 mg once weekly
Weekly dosing 6:
0.3, 0.3, 0.6, 0.6, 0.9, 0.9, 1.2, 1.2, 1.5, 1.5, 1.8, 1.8, 2.1, 2.1, 2.4, 2.4 mg twice weekly

### Mode of Administration:

Subcutaneous injection

### Comparator Product:

Placebo
Dose: Not applicable
Mode of administration: Subcutaneous injection

### Duration of Treatment

6 weeks (Part A) and 16 weeks (Part B)

### Clinical Pharmacology

The primary endpoint of this trial is described in the safety section below.

Secondary endpoints were Cₘₐₓ after the first dose and AUC₀₋₁₆₈. Secondary endpoints were only applicable for the weekly dosing schemes.

### Safety Criteria for Evaluation

The primary endpoint to assess safety and tolerability of Compound I was the cumulative number of patients withdrawn from up-titration by up-titration scheme.

Further criteria of interest:
AEs (including clinically relevant findings from the physical examination and assessment of local tolerability)
Safety laboratory tests
12-lead ECG
Vital signs (blood pressure, pulse rate)
Continuous ECG (including mean daily heart rate)
Assessment of local tolerability by the investigator
The cumulative number [N (%)] of patients withdrawn from up-titration by up-titration scheme and week

### Statistical Methods

Descriptive statistics were calculated for all endpoints. For the primary endpoint, exploratory 95% CIs for the proportions were calculated using normal approximation. Exploratory 95% CIs for the difference of proportions comparing up-titration schemes were calculated. Dose proportionality of Compound I was explored using a regression model. No formal interim analysis was conducted. Data from the completed 6-week cohorts were analysed before database lock for internal decision-making.

### SUMMARY—CONCLUSIONS

Trial Patients and Compliance with the Clinical Trial Protocol

### Part A

Eighty patients were treated. Ten patients (12.5%) prematurely discontinued trial medication, most commonly due to an adverse event (6 patients, 7.5%). Part A of the trial was conducted in male patients only. All but 2 of the patients were White. None of the patients were reported as ‘Hispanic or Latino’. All patients were categorised as obese or overweight. An overview of the demographic characteristics is presented in Table 1 below. No important protocol deviations were reported.

TABLE 1

| | Demographic characteristics, Part A | | | | | |
|---|---|---|---|---|---|---|
| | Placebo | Daily | Weekly 1 | Weekly 2 | Weekly 3 | Total |
| Number of patients, | 13 (100.0) | 15 (100.0) | 17 (100.0) | 18 (100.0) | 17 (100.0) | 80 (100.0) |
| N (%) | | | | | | |
| Male patients, N (%) | 13 (100.0) | 15 (100.0) | 17 (100.0) | 18 (100.0) | 17 (100.0) | 80 (100.0) |
| Race, N (%) | | | | | | |
| White | 12 (92.3) | 15 (100.0) | 16 (94.1) | 18 (100.0) | 17 (100.0) | 78 (97.5) |
| Black or African | 0 | 0 | 1 (5.9) | 0 | 0 | 1 (1.3) |
| American | | | | | | |
| Native Hawaiian or | 1 (7.7) | 0 | 0 | 0 | 0 | 1 (1.3) Pacific |
| other Islander | | | | | | |
| Mean age | | | | | | |
| (SD) [years] | 53.2 (8.5) | 48.1 (7.7) | 42.6 (11.8) | 49.6 (9.7) | 39.1 (12.6) | 46.2 (11.3) |
| Mean weight [kg] | 92.72 | 98.02 | 97.88 | 95.17 | 97.38 | 96.35 |
| (SD) | (6.16) | (9.28) | (10.79) | (12.43) | (9.69) | (10.03) |
| Mean BMI [kg/m²] | 29.71 | 30.70 | 30.30 | 30.33 | 30.28 | 30.28 |
| (SD) | (1.61) | (2.85) | (2.47) | (2.90) | (2.32) | (2.46) |

The planned total dose of Compound I per patient based on pre-specified up-titration was 10.50 mg (Daily group), 6.30 mg (Weekly 1), 8.40 mg (Weekly 2), and 9.60 mg (Weekly 3). The actual mean total dose of per patient was 10.5 mg (SD 0.0) for the Daily dosing group, 4.75 mg (SD 2.04) for Weekly 1, 6.38 mg (SD 2.58) for Weekly 2, and 6.85 mg (SD 2.53) for Weekly 3.

### Primary Endpoint

The proportion of patients who withdrew from up-titration progressively increased from the Daily dosing group (0 patients) to Weekly 1 (6 patients 35.3%), Weekly 2 (8 patients, 44.4%), and Weekly 3 (13 patients, 76.5%). Overall, 27 patients (40.3%) treated with Compound I withdrew from planned up-titration (Table 2).

TABLE 2
Stop of up-titration by dosing group, Part A

| | | Patients withdrawn from | 95% CI for the |
|---|---|---|---|
| Dosing group | Patients | up-titration, N (%) | proportion |
| Placebo | 13 | 2 (15.4) | (0.000, 34.998) |
| Daily | 15 | 0 | — |
| Weekly 1 | 17 | 6 (35.3) | (12.577, 58.011) |
| Weekly 2 | 18 | 8 (44.4) | (21.489, 67.400) |
| Weekly 3 | 17 | 13 (76.5) | (56.307, 96.635) |
| Compound I | 67 | 27 (40.3) | (28.554, 52.043) |
| Total | | | |

### Part B

Forty-five patients were treated. Eight patients (17.8%) prematurely discontinued trial medication, most commonly due to an adverse event (6 patients, 13.3%).

The majority of the patients were male. All patients were White and all were categorised as obese or overweight. None of the patients were reported as ‘Hispanic or Latino’. An overview of the demographic characteristics is presented in Table 3 below. No important protocol deviations were reported.

TABLE 3
Demographic characteristics, Part B

| | Placebo | Weekly 4 | Weekly 5 | Weekly 6 | Total |
|---|---|---|---|---|---|
| Number of patients, N (%) | 9 (100.0) | 13 (100.0) | 11 (100.0) | 12 (100.0) | 45 (100.0) |
| Sex, N (%) | | | | | |
| Male | 6 (66.7) | 9 (69.2) | 7 (63.6) | 9 (75.0) | 31 (68.9) |
| Female | 3 (33.3) | 4 (30.8) | 4 (36.4) | 3 (25.0) | 14 (31.1) |
| White patients | 9 (100.0) | 13 (100.0) | 11 (100.0) | 12 (100.0) | 45 (100.0) |
| Mean age, (SD) [years] | 44.6 (10.3) | 45.0 (16.8) | 48.9 (14.0) | 42.2 (12.7) | 45.1 (13.7) |
| Mean weight (SD) [kg] | 101.02 (17.15) | 87.65 (9.92) | 94.15 (19.16) | 95.75 (12.58) | 94.07 (15.04) |
| Mean BMI (SD) [kg/m²] | 32.33 (4.28) | 29.64 (1.92) | 30.45 (3.66) | 31.68 (3.69) | 30.92 (3.45) |

The planned total dose of Compound I per patient based on pre-specified up-titration was 24.00 mg (Weekly 4), 43.20 mg (Weekly 5), and 43.20 mg (Weekly 6). The mean actual total dose per patient was 16.39 mg (9.75) for Weekly 4, 38.24 mg (SD 11.05) for Weekly 5, and 42.60 mg (2.08) for Weekly 6.

### Primary Endpoint

The proportion of patients who withdrew from up-titration was highest for the Weekly 5 dosing group (4 patients, 36.4%) and lowest for the Weekly 6 group (1 patient, 8.3%). Overall, 8 patients (22.2%) treated with Compound I withdrew from planned up-titration (Table 4).

TABLE 4
Stop of up-titration by dosing group, Part B

| | | Patients withdrawn from | 95% CI for the |
|---|---|---|---|
| Dosing group | Patients | up-titration, N (%) | proportion |
| Placebo | 9 | 0 | — |
| Weekly 4 | 13 | 3 (23.1) | (0.174, 45.980) |
| Weekly 5 | 11 | 4 (36.4) | (7.936, 64.791) |
| Weekly 6 | 12 | 1 (8.3) | (0.000, 23.971) |
| Compound I | 36 | 8 (22.2) | (8.642, 35.803) |
| Total | | | |
| Total | 45 | 8 (17.8) | (6.607, 28.948) |

### Clinical Pharmacology Results

Selected PK parameters of Compound I are displayed in Table 5. Following subcutaneous administration of Compound I, the gMean plasma Cₘₐₓ and AUC₀₋₁₆₈ increased with further weekly dosing: In weekly dose groups 1 and 2 by escalating the weekly dose and in weekly dose group 3 in addition due to accumulation (first two weeks). The same holds true for the 16 weeks dose groups. Cₘₐₓ and AUC₀₋₁₆₈ increased on one hand by escalating the weekly dose and on the other hand by accumulation after application of similar subsequent doses.

TABLE 5
Plasma pharmacokinetic parameters of Compound I after subcutaneous
administration of different doses of Compound I of different up-titration schemes
Weekly up-titration scheme 1

| Week | 1 | | | 2 | | | 3 | | | 4 | | | 5 | | | 6 | | |
|---|---|---|---|---|---|---|---|---|---|---|---|---|---|---|---|---|---|---|
| Dose | 0.3 mg | | | 0.6 mg | | | 0.9 mg | | | 1.2 mg | | | 1.5 mg | | | 1.8 mg | | |
| | | | gCV | | | gCV | | | gCV | | | gCV | | | gCV | | | gCV |
| | N | gMean | [%] | N | gMean | [%] | N | gMean | [%] | N | gMean | [%] | N | gMean | [%] | N | gMean | [%] |
| AUC¹ | 11 | 617 | 16.6 | 15 | 1410 | 16.2 | 12 | 2480 | 10.9 | 10 | 3360 | 10.5 | 10 | 4490 | 9.79 | 9 | 5530 | 10.7 |
| Cmax | 16 | 5.08 | 25.0 | 16 | 12.3 | 25.5 | 13 | 20.6 | 15.0 | 10 | 28.1 | 11.0 | 10 | 37.8 | 15.1 | 9 | 45.5 | 13.2 |
| [nM] | | | | | | | | | | | | | | | | | | |

Weekly up-titration scheme 2

| Week | 1 | | | 2 | | | 3 | | | 4 | | | 5 | | | 6 | | |
|---|---|---|---|---|---|---|---|---|---|---|---|---|---|---|---|---|---|---|
| Dose | 0.4 mg | | | 0.8 mg | | | 1.2 mg | | | 1.6 mg | | | 2.0 mg | | | 2.4 mg | | |
| | | | gCV | | | gCV | | | gCV | | | gCV | | | gCV | | | gCV |
| | N | gMean | [%] | N | gMean | [%] | N | gMean | [%] | N | g Mean | [%] | N | g Mean | [%] | N | gMean | [%] |
| AUC¹ | 12 | 818 | 20.0 | 14 | 2040 | 17.2 | 12 | 3560 | 13.1 | 10 | 5130 | 15.7 | 8 | 6550 | 11.6 | 8 | 8190 | 12.8 |
| Cmax | 16 | 6.51 | 32.2 | 15 | 16.9 | 23.4 | 13 | 31.7 | 24.9 | 10 | 44.3 | 19.2 | 8 | 57.7 | 10.3 | 8 | 70.6 | 14.8 |
| [nM] | | | | | | | | | | | | | | | | | | |

Weekly up-titration scheme 3

| Week | 1 | | | 2 | | | 3 | | | 4 | | | 5 | | | 6 | | |
|---|---|---|---|---|---|---|---|---|---|---|---|---|---|---|---|---|---|---|
| Dose | 0.6 mg | | | 0.6 mg | | | 1.2 mg | | | 1.8 mg | | | 2.4 mg | | | 3.0 mg | | |
| | | | gCV | | | gCV | | | gCV | | | lgCV | | | gCV | | | gCV |
| | N | gMean | [%] | N | gMean | [%] | N | gMean | [%] | N | gMean | [%] | N | gMean | [%] | N | gMean | [%] |
| AUC¹ | 13 | 1240 | 15.8 | 13 | 1710 | 17.5 | 11 | 3230 | 11.6 | 9 | 5200 | 15.7 | 6 | 7190 | 8.88 | 2 | 10400 | 13.7 |
| Cmax | 16 | 10.7 | 23.7 | 15 | 14.4 | 22.2 | 13 | 28.1 | 15.3 | 10 | 46.5 | 18.1 | 6 | 63.0 | 7.48 | 2 | 88.7 | 8.70 |
| [nM] | | | | | | | | | | | | | | | | | | |

Weekly up-titration scheme 4

| Week | 1 | | | 3 | | | 5 | | | 6 | | | 7 | | | 19 | | |
|---|---|---|---|---|---|---|---|---|---|---|---|---|---|---|---|---|---|---|
| Dose | 0.6 mg | | | 0.6 mg | | | 1.2 mg | | | 1.2 mg | | | 1.2 mg | | | 1.8 mg | | |
| | | | gCV | | | gCV | | | gCV | | | gCV | | | gCV | | | gCV |
| | N | gMean | [%] | N | g Mean | [%] | N | gMean | [%] | N | gMean | [%] | N | gMean | [%] | N | gMean | [%] |
| AUC¹ | 12 | 1570 | 23.3 | 10 | 2490 | 16.1 | 7 | 3940 | 17.0 | 9 | 4700 | 14.3 | 9 | 5190 | 17.1 | 9 | 6660 | 15.0 |
| Cmax | 13 | 15.7 | 39.5 | 11 | 22.3 | 23.5 | 10 | 36.0 | 16.0 | 10 | 41.9 | 15.8 | 10 | 49.0 | 26.2 | 10 | 60.2 | 18.0 |
| [nM] | | | | | | | | | | | | | | | | | | |
| Weekly up-titration scheme 4 | | | | | | | | | | | | | | | | | | |
| Week | 11 | | | 13 | | | 15 | | | 16 | | | | | | | | |
| Dose | 1.8 mg | | | 2.4 mg | | | 2.4 mg | | | 2.4 mg | | | | | | | | |
| | | | gCV | | | gCV | | | gCV | | | gCV | | | | | | |
| | N | gMean | [%] | N | gMean | [%] | N | gMean | [%] | N | gMean | [%] | | | | | | |
| AUC¹ | 7 | 8160 | 15.7 | 6 | 10300 | 22.2 | 5 | 10900 | 23.8 | 6 | 11500 | 20.9 | | | | | | |
| Cmax | 9 | 69.9 | 16.4 | 7 | 88.3 | 22.2 | 7 | 87.1 | 22.1 | 7 | 97.3 | 21.7 | | | | | | |
| [nM] | | | | | | | | | | | | | | | | | | |

Weekly up-titration scheme 5

| Week | 1 | | | 3 | | | 5 | | | 6 | | | 7 | | | 9 | | |
|---|---|---|---|---|---|---|---|---|---|---|---|---|---|---|---|---|---|---|
| Dose | 0.6 mg | | | 1.2 mg | | | 1.8 mg | | | 1.8 mg | | | 2.4 mg | | | 3.0 mg | | |
| | | | gCV | | | gCV | | | gCV | | | gCV | | | gCV | | | gCV |
| | N | gMean | [%] | N | gMean | [%] | N | gMean | [%] | N | gMean | [%] | N | gMean | [%] | N | gMean | [%] |
| AUC¹ | 8 | 1440 | 34.1 | 10 | 3790 | 27.7 | 8 | 6080 | 25.7 | 10 | 6770 | 27.8 | 9 | 9080 | 27.3 | 9 | 11300 | 24.8 |
| Cmax | 11 | 13.2 | 38.9 | 11 | 31.5 | 31.7 | 10 | 57.9 | 25.7 | 10 | 63.3 | 29.7 | 10 | 77.8 | 29.0 | 10 | 102 | 30.1 |
| [nM] | | | | | | | | | | | | | | | | | | |
| Weekly up-titration scheme 5 | | | | | | | | | | | | | | | | | | |
| Week | 11 | | | 13 | | | 15 | | | 16 | | | | | | | | |
| Dose | 3.6 mg | | | 4.2 mg | | | 4.8 mg | | | 4.8 mg | | | | | | | | |
| | | | gCV | | | gCV | | | gCV | | | gCV | | | | | | |
| | N | gMean | [%] | N | gMean | [%] | N | gMean | [%] | N | gMean | [%] | | | | | | |
| AUC¹ | 9 | 14200 | 28.5 | 6 | 15900 | 23.5 | 6 | 18700 | 24.7 | 6 | 19700 | 23.5 | | | | | | |
| Cmax | 9 | 129 | 26.6 | 7 | 134 | 26.4 | 7 | 161 | 25.4 | 7 | 193 | 15.8 | | | | | | |
| [nM] | | | | | | | | | | | | | | | | | | |

twice weekly up-titration scheme

| Week | 1 | | | 3 | | | 5 | | | 6 | | | 7 | | | 9 | | |
|---|---|---|---|---|---|---|---|---|---|---|---|---|---|---|---|---|---|---|
| Dose | 0.3 mg | | | 0.6 mg | | | 0.9 mg | | | 0.9 mg | | | 1.2 mg | | | 1.5 mg | | |
| | | | gCV | | | gCV | | | gCV | | | gCV | | | gCV | | | gCV |
| | N | gMean | [%] | N | gMean | [%] | N | gMean | [%] | N | gMean | [%] | N | gMean | [%] | N | gMean | [%] |
| AUC0-72 | 5 | 342 | 22.8 | 8 | 1200 | 17.9 | 9 | 2390 | 21.2 | 12 | 2780 | 25.5 | 10 | 3560 | 16.2 | 9 | 5000 | 18.5 |
| [nM*h] | | | | | | | | | | | | | | | | | | |
| Cmax | 12 | 5.38 | 26.2 | 12 | 20.4 | 22.0 | 12 | 40.8 | 19.7 | 12 | 49.2 | 28.1 | 12 | 58.8 | 18.1 | 12 | 78.3 | 25.2 |
| [nM] | | | | | | | | | | | | | | | | | | |
| twice weekly up-titration scheme | | | | | | | | | | | | | | | | | | |
| Week | 11 | | | 13 | | | 15 | | | 16 | | | | | | | | |
| Dose | 1.8 mg | | | 2.1 mg | | | 2.4 mg | | | 2.4 mg | | | | | | | | |
| AUC0-72 | 12 | 5690 | 23.0 | 8 | 6700 | 25.9 | 9 | 7620 | 19.8 | 10 | 8550 | 16.4 | | | | | | |
| [nM*h] | | | | | | | | | | | | | | | | | | |
| Cmax | 12 | 93.6 | 21.9 | 11 | 108 | 22.0 | 11 | 132 | 25.0 | 11 | 142 | 16.0 | | | | | | |
| [nM] | | | | | | | | | | | | | | | | | | |

¹AUC₀₋₁₆₈ [nM*h]

In all weekly up-titration schemes (including twice weekly) the gMean values for the plasma Compound I AUC₀₋₁₆₈ ranged from 617 nmol·h/L for the first 0.3 mg dose of weekly dose group 1 up to 19700 nmol·h/L for the last 4.8 mg dose in the weekly up-titration scheme 5.

In all weekly up-titration schemes (including twice weekly) the gMean values for the plasma Compound I Cₘₐₓ ranged from 5.08 nmol/L for the first 0.3 mg dose of weekly dose group 1 up to 193 nmol/L for the last 4.8 mg dose in the weekly up-titration scheme 5.

### Safety Results

### Part A

All patients in the Compound I dosing groups were reported with adverse events that were assessed as related to study drug by the investigator. Three patients (3.8%, all in the Compound I) had events of severe intensity, while 6 patients (7.5%) had AEs leading to treatment discontinuation. One patient was reported with a serious AE (Table 6; see details regarding this event below). No patients died.

TABLE 6
Overall summary of adverse events, Part A

| | Placebo | | | | | | |
|---|---|---|---|---|---|---|---|
| | Total | Daily | Weekly 1 | Weekly 2 | Weekly 3 | Cmp I | Total |
| Total Number of patients | | | | | | | |
| N (100%) | 13 | 15 | 17 | 18 | 17 | 67 | 80 |
| Patients with any AE | 10 (76.9) | 15 (100) | 17 (100) | 18 (100) | 17 (100) | 67 (100) | 77 (96.3) |
| N (%) | | | | | | | |
| Severe | 0 | 0 | 1 (5.9) | 2 (11.1) | 0 | 3 (4.5) | 3 (3.8) |
| Investigator defined | 9 (69.2) | 15 (100) | 17 (100) | 18 (100) | 17 (100) | 67 (100) | 76 (95.0) |
| drug-related | | | | | | | |
| Leading to discontinuation | 1 (7.7) | 0 | 1 (5.9) | 2 (11.1) | 2 (11.8) | 5 (7.5) | 6 (7.5) |
| of trial drug | | | | | | | |
| Serious | 0 | 0 | 0 | 1 (5.6) | 0 | 1 (1.5) | 1 (1.3) |
| Other significant AEs | 1 (7.7) | 0 | 1 (5.9) | 2 (11.1) | 2 (11.8) | 5 (7.5) | 6 (7.5) |
| (ICH E3) | | | | | | | |

The most common AEs by SOC (system organ class) were gastrointestinal disorders (82.5% overall). Other types of AEs reported for at least 20% of patients overall were metabolism and nutrition disorders (66.3%), ‘general disorders and administration site conditions’ (52.5%), nervous system disorders (45.0%), cardiac disorders (27.5%), and infections and infestations (22.5%). Two patients had findings based on local tolerability of the injection site (Daily group and placebo group).

The most common drug-related AEs were nausea and decreased appetite (each 62.5%). Other related events reported for at least 20% of patients were early satiety (41.3%), dyspepsia (33.8%), abdominal distension (32.5%), headache (26.3%), diarrhoea (25.0%), and vomiting (21.3%). No drug-related cardiac disorders were reported in the Daily dosing group, while the frequency ranged from 29.4 to 47.1% in the other Compound I groups. In addition, there were fewer drug-related cases in the Daily dosing group than in the other Compound I groups for abdominal distension (Daily: 13.3%; other Compound I groups: range 38.9 to 52.9%) and vomiting (Daily: no cases; other Compound I groups: range 23.5 to 38.9%).

Three patients (3.8%) were reported with AEs of severe intensity. Of these, 2 patients were reported with severe diarrhoea (Weekly 1 and Weekly 2) and one patient was reported with severe vomiting (Weekly 2).

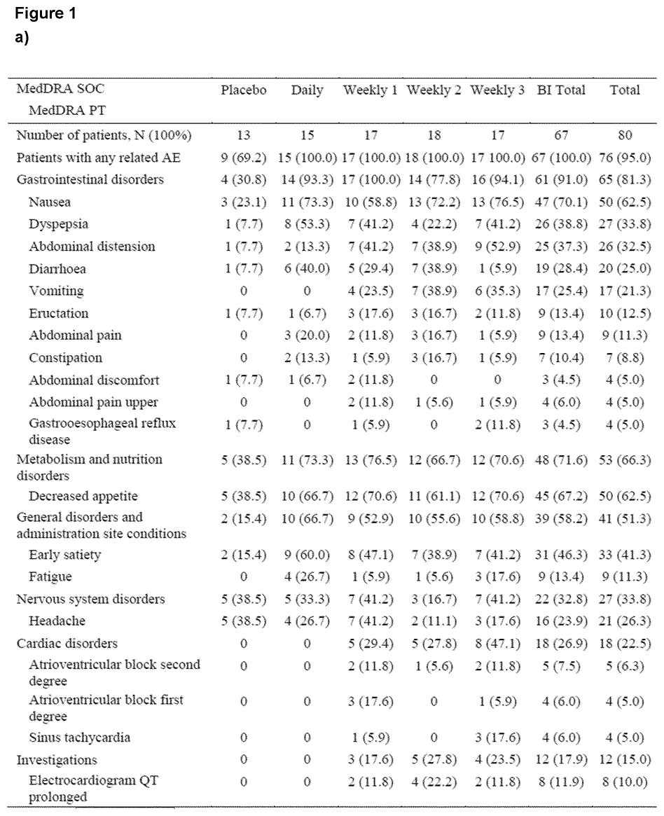

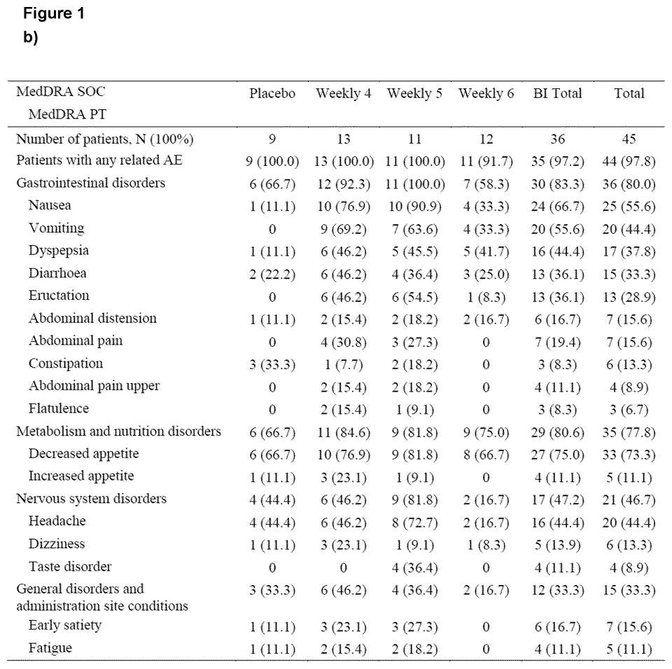

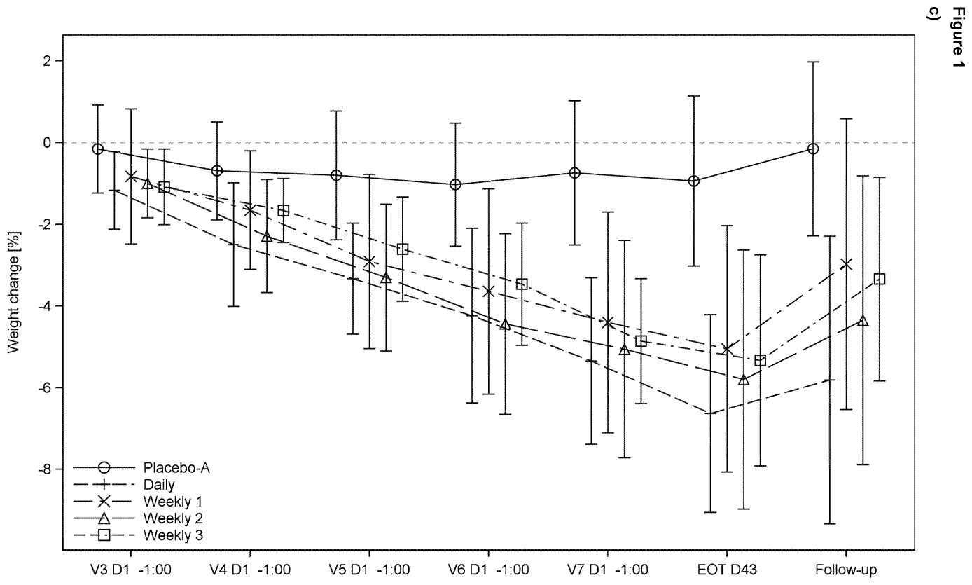

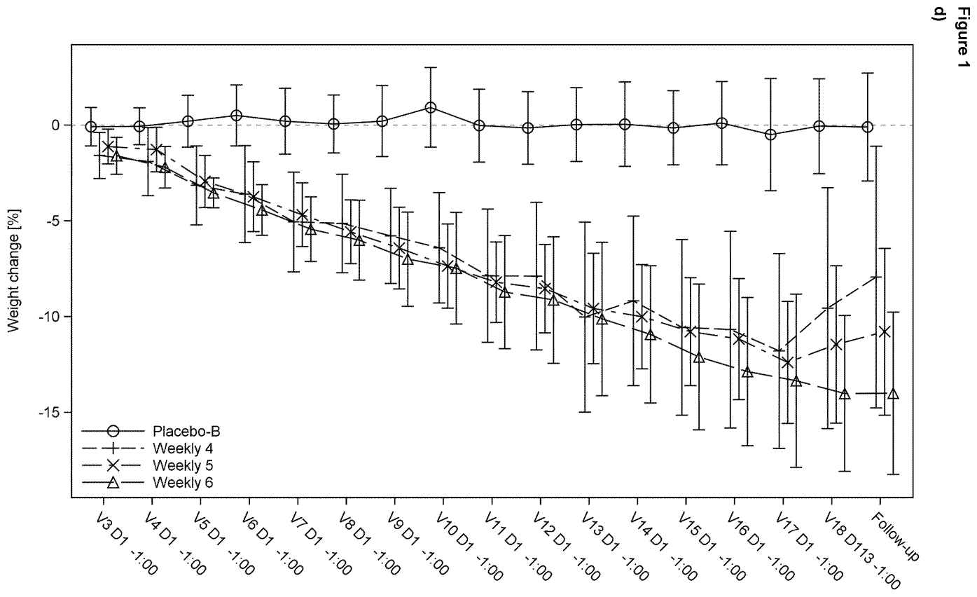

### See FIG. 1 a)

One patient was reported with a serious AE (ventricular tachycardia, Weekly 2 group). The event was assessed by the investigator as related to study drug and led to treatment discontinuation. The patient recovered from the event on the day of its onset. The investigator had initially reported the event as non-serious. The event was later re-categorised as serious, since the preferred term ventricular tachycardia had been added to Sponsor's list of ‘Always Serious Events’ after the initial reporting of the event. There were no treatment-emergent clinically relevant findings in the clinical laboratory analyses. Continuous ECG monitoring over 24 hours revealed increases in mean heart rate in all Compound I treatment groups.

### Part B

Most patients in the Compound I dosing groups were reported with adverse events that were assessed as related to study drug by the investigator (97.8%). AEs of severe intensity were reported for 22.2% of patients, while 13.3% of patients had AEs leading to treatment discontinuation. No serious AEs were reported (Table 7). No patients died.

TABLE 7
Overall summary of adverse events, Part B

| | Placebo | Weekly 4 | Weekly 5 | Weekly 6 | Cmp I Total | Total |
|---|---|---|---|---|---|---|
| Number of patients, N (100%) | 9 | 13 | 11 | 12 | 36 | 45 |
| Patients with any AE, N (%) | 9 (100.0) | 13 (100.0) | 11 (100.0) | 11 (91.7) | 35 (97.2) | 44 (97.8) |
| Severe | 2 (22.2) | 4 (30.8) | 4 (36.4) | 0 | 8 (22.2) | 10 (22.2) |
| Investigator defined drug-related | 9 (100.0) | 13 (100.0) | 11 (100.0) | 11 (91.7) | 35 (97.2) | 44 (97.8) |
| Leading to discontinuation of | 0 | 3 (23.1) | 2 (18.2) | 1 (8.3) | 6 (16.7) | 6 (13.3) |
| trial drug | | | | | | |
| Serious | 0 | 0 | 0 | 0 | 0 | 0 |
| Other significant AEs (ICH E3) | 0 | 4 (30.8) | 3 (27.3) | 1 (8.3) | 8 (22.2) | 8 (17.8) |

The most common AEs by SOC were gastrointestinal disorders (80.0%). Other types of AEs reported for at least 20% of patients overall were metabolism and nutrition disorders (77.8%), nervous system disorders (51.1%), infections and infestations (44.4%), ‘general disorders and administration site conditions’ (42.2%), and ‘musculoskeletal and connective tissue disorders’ (22.2%). Three patients had findings based on local tolerability of the injection site (Weekly 5 and Weekly 6 group).

### See FIG. 1 b)

The most common drug-related AE was decreased appetite (73.3%). Other events reported for at least 20% of patients were nausea (55.6%), headache (44.4%), vomiting (44.4%), dyspepsia (37.8%), diarrhoea (33.3%), and eructation (28.9%). Drug-related gastrointestinal disorders were less frequent in the Weekly 6 group (58.3%) than in Weekly 4 group (92.3%) and Weekly 5 group (all patients). There were no cases of drug-related cardiac disorders in Part B.

Ten patients (22.2%) were reported with AEs of severe intensity, the majority of which were gastrointestinal disorders (9 patients, 20.0%).

There were no treatment-emergent clinically relevant findings in the clinical laboratory analyses. ECG analyses revealed increases in heart rate in all Compound I treatment groups.

### CONCLUSIONS

Overall, the investigation of the daily and weekly up-titration schemes of Compound I in patients with overweight or obesity revealed no unexpected safety or tolerability concerns.

The majority of patients in the Compound I dosing groups were reported with gastrointestinal disorder, most commonly nausea. In the Daily dosing scheme, no drug-related cardiac disorders were reported and certain gastrointestinal disorders were notably reduced compared with the Weekly dosing schemes over 6 weeks. No drug-related cardiac disorders were reported for the 16-week dosing schemes. The twice-weekly dosing scheme (Weekly 6) showed the lowest frequency of drug-related gastrointestinal disorders among the 16-week dosing schemes. Following administration of Compound I, exposures in terms of Cₘₐₓ and AUC₀₋₁₆₈ increased with increasing doses or accumulated after application of similar subsequent doses.

### Further Endpoint—Body Weight

The effect of different doses including the different dose escalation schemes of Compound I on body weight was evaluated before (baseline) and at different time points after Compound I has been dosed for 6 weeks (Part A) and 16 weeks (Part B). Baseline is defined as last measurement prior to first administration of Compound I. Body weight change was evaluated in two ways: absolute change from baseline (kg) and change from baseline in percent (%). Arithmetic mean body weight decreased after administration of Compound I, both in terms of absolute and percent change after 6 (FIG. 1 c)) and after 16 weeks treatment (FIG. 1 d)).

In the follow-up period body weight was again increasing slowly after the end of the treatment in part A and in part B in the weekly dose groups 4 and 5.

Placebo-corrected statistical analysis revealed a maximum % decrease of mean body weight at the end of trial (EOT; Day 43) with −5.79±1.05% in the daily dose group, −4.22±1.02% in the weekly 1 dose group, −4.92±1.00% in the weekly 2 dose group and −4.48±1.02% in the weekly 3 dose group after 6 weeks treatment (Part A).

The maximum placebo corrected weight loss after 16 weeks of treatment (Part B) could be detected at Day 113 with −9.03±1.63% in the dose group weekly 4, −11.2±1.64% in the weekly group 5 and −13.8±1.60% in the weekly dose group 6.

As described above due to tolerability reasons the patients were allowed to stop dose-escalation at any time and remain at the previous dose. It is therefore not possible to discriminate a clear dose dependency between the different treatment groups as there is overlap of the doses, which had been taken by the patients in the different groups.

### Example 2: A Study to Test Different Doses of Compound I in Healthy Japanese Men

A phase I, single-blinded, randomized, multiple dose, placebo-controlled study to assess the safety, tolerability, pharmacokinetics and pharmacodynamics of different dose escalation schemes of Compound I in healthy Japanese male subjects with BMI 23-40 kg/m²

### Objectives

Main objectives were to investigate the safety, tolerability, pharmacokinetics (PK) and pharmacodynamics (PD) of different dose escalation schemes of Compound I in healthy Japanese male subjects with BMI 23 to 40 kg/m² and to determine a dose escalation scheme for future studies.

### Methodology

This study was a randomised, placebo-controlled, single-blind, and parallel-group clinical trial with up to 3 dose-escalation schemes (Dose Groups [DGs]1 to 3) in healthy Japanese male subjects with BMI 23 to 40 kg/m². The weekly (DG1 and DG2) or twice a week (DG3) dosing in this scheme allowed for immediate clinical evaluation whether a further increase in the cumulative weekly dose was safe and well tolerated. The treatment duration was 16 weeks in total including a dose escalation phase to minimize Gastrointestinal (GI) Adverse Events (AEs) of Compound I. This was followed by a 4-week follow-up period.

### Number of Subjects

Planned: Entered: 36
Actual: Entered: 37
   Compound I DG1:
   Entered: 10 Treated: 9 Analysed (for primary endpoint): 9
   Compound I DG2:
   Entered: 9 Treated: 9 Analysed (for primary endpoint): 9
   Compound I DG3:
   Entered: 9 Treated: 9 Analysed (for primary endpoint): 9
   Placebo (matching Compound I):
   Entered: 9 Treated: 9 Analysed (for primary endpoint): 9

### Diagnosis

Not applicable

Main criteria for Inclusion

Healthy Japanese male volunteers aged from 20 to 45 years with a 3-month stable (defined as no more than 5% change) BMI of 23 to 40 kg/m², a minimum absolute body weight of 65 kg, and HbA1c<6.5% were included.

Compound I solution for injection (2 mg/mL)

### Doses: 16-Week Up-Titration

DG1: 0.3, 0.6, 0.9, 1.2, 1.5, 1.8, 1.8, 1.8, 1.8, 1.8, 1.8, 1.8, 1.8, 1.8, 1.8, 1.8 mg once weekly
DG2: 0.6, 0.6, 1.2, 1.2, 1.8, 1.8, 2.4, 2.4, 3.0, 3.0, 3.6, 3.6, 4.2, 4.2, 4.8, 4.8 mg once weekly
DG3: 0.3, 0.3, 0.6, 0.6, 0.9, 0.9, 1.2, 1.2, 1.5, 1.5, 1.8, 1.8, 2.1, 2.1, 2.4, 2.4 mg twice weekly

### Mode of Administration:

Subcutaneous injection

### Comparator Product:

Placebo (matching Compound I)
Dose: Not applicable
Mode of administration: Subcutaneous injection

### Duration of Treatment

16 weeks of treatment followed by 4-week follow-up after trial drug termination

### Clinical Pharmacology/Other Criteria for Evaluation

The primary endpoint in this trial was a safety endpoint and is described in the safety section below.

The secondary endpoints were area under the concentration-time curve of the analyte in plasma over the time interval from 0 to 168 h (AUC₀₋₁₆₈) and maximum measured concentration of the analyte in plasma (Cₘₐₓ) after the first dose.

### Safety Criteria for Evaluation

The primary endpoint to assess the safety and tolerability of Compound I was the cumulative percentage (%) of subjects withdrawn from the up-titration by the dose escalation scheme.

### Further Criteria of Interest:

AEs (including clinically relevant findings from the physical examination)
Safety laboratory tests
12-lead electrocardiogram (ECG)
Continuous ECG monitoring
Vital signs (blood pressure, pulse rate)
Columbia-Suicide Severity Rating Scale (C-SSRS)
Change from baseline in heart rate following treatment with Compound I relative to placebo (until 24 hours after Compound I administration)
The cumulative number [N (%)] of subjects withdrawn from the up-titration by the dose escalation scheme and week

### Statistical Methods

The main objective of this trial was to assess the safety and tolerability of Compound I as well as PK and PD parameters by using descriptive statistics for all endpoints, which were compared across treatment groups. Further analysis comprised linear modelling for attainment of steady state in DG1.

### Summary—Conclusions

Trial Subjects and Compliance with the Clinical Trial Protocol

A total of 37 Japanese male volunteers were randomised, and 36 subjects were treated in the trial. One subject was randomised but discontinued the trial prior to the initiation of the trial medication because of an AE. Two subjects (5.6% of 36 treated subjects) in DG1 prematurely discontinued from trial medication because of AEs. All subjects completed the planned observation time.

All of the 36 subjects in this trial were male and Asian; more precisely Japanese. The mean age (SD) was 34.2 (7.6) years and mean BMI (SD) was 25.19 (1.77) kg/m².

Most of the demographic characteristics were well balanced across the groups. An overview of the demographic data is presented in Table 8 below.

TABLE 8
Demographic data-TS

| | | | Compound I | | | | | | | |
|---|---|---|---|---|---|---|---|---|---|---|
| | Placebo | | DG1 | | DG2 | | DG3 | | Total | |
| Number of subjects, | 9 | (100.0) | 9 | (100.0) | 9 | (100.0) | 9 | (100.0) | 36 | (100.0) |
| N (%) | | | | | | | | | | |
| Sex, N (%) | | | | | | | | | | |
| Male | 9 | (100.0) | 9 | (100.0) | 9 | (100.0) | 9 | (100.0) | 36 | (100.0) |
| Race, N (%) | | | | | | | | | | |
| Asian | 9 | (100.0) | 9 | (100.0) | 9 | (100.0) | 9 | (100.0) | 36 | (100.0) |
| Ethnicity, N (%) | | | | | | | | | | |
| Not Hispanic/ | 9 | (100.0) | 9 | (100.0) | 9 | (100.0) | 9 | (100.0) | 36 | (100.0) |
| Latino | | | | | | | | | | |
| Mean age (SD) | 36.2 | (8.5) | 37.0 | (8.0) | 31.9 | (5.8) | 31.6 | (7.5) | 34.2 | (7.6) |
| [years] | | | | | | | | | | |
| Mean weight (SD) | 76.56 | (7.08) | 73.78 | (6.80) | 72.17 | (5.25) | 77.76 | (7.85) | 75.06 | (6.89) |
| [kg] | | | | | | | | | | |
| Mean BMI (SD) | 25.89 | (2.00) | 25.59 | (1.85) | 24.14 | (0.82) | 25.13 | (1.92) | 25.19 | (1.77) |
| [kg/m²] | | | | | | | | | | |

The planned total dose of Compound I per subject based on the pre-specified up-titration was 24.30 mg (DG1), 43.20 mg (DG2), and 43.20 mg (DG3). The mean actual total dose per subject was 21.067 mg (SD 7.883) for DG1, 30.667 mg (SD 10.583) for DG2, and 42.933 mg (SD 0.529) for DG3.

One important protocol deviation was reported for 1 subject receiving placebo within the first dosing scheme cohort. Full-volume administration of planned dose (1.8 mg) was not done because of leak from the syringe.

### Clinical Pharmacology Results

Specified PK parameters of Compound I, AUC₀₋₁₆₈ and Cₘₐₓ, are displayed in Table 9; these parameters are pre-specified secondary endpoint of this trial. In Table 9, only the gMean PK parameters of subjects who followed the planned dose rising scheme are included. Following subcutaneous administration of Compound I, the gMean plasma AUC₀₋₁₆₈ and Cₘₐₓ increased with further weekly or twice weekly dosing.

TABLE 9
Pharmacokinetic parameters of Compound I after subcutaneous
administration of different doses of Compound I in DGs 1 to 3 (only data with planned dose)
DG1 (weekly administration)

| Week | 1 | | | 3 | | | 5 | | | 6 | | | 7 | | | 9 | | |
|---|---|---|---|---|---|---|---|---|---|---|---|---|---|---|---|---|---|---|
| Dose | 0.3 mg | | | 0.9 mg | | | 1.5 mg | | | 1.8 mg | | | 1.8 mg | | | 1.8 mg | | |
| | | | gCV | | | gCV | | | gCV | | | gCV | | | gCV | | | gCV |
| | N | gMean | [%] | N | gMean | [%] | N | gMean | [%] | N | gMean | [%] | N | gMean | [%] | N | gMean | [%] |
| AUC¹ | — | — | — | 8 | 3620 | 14.0 | 8 | 6740 | 14.2 | 7 | 8410 | 16.3 | 6 | 9010 | 15.1 | 7 | 8520 | 6.13 |
| Cmax | 9 | 3.20 | 25.2 | 8 | 32.8 | 20.1 | 8 | 64.4 | 19.6 | 7 | 79.0 | 20.5 | 7 | 79.4 | 33.9 | 7 | 79.0 | 11.4 |
| [nM/L] | | | | | | | | | | | | | | | | | | |
| DG1 (weekly administration) | | | | | | | | | | | | | | | | | | |
| Week | 11 | | | 13 | | | 15 | | | 16 | | | | | | | | |
| Dose | 1.8 mg | | | 1.8 mg | | | 1.8 mg | | | 1.8 mg | | | | | | | | |
| | | | gCV | | | gCV | | | gCV | | | gCV | | | | | | |
| | N | gMean | [%] | N | gMean | [%] | N | gMean | [%] | N | gMean | [%] | | | | | | |
| AUC¹ | 7 | 8830 | 8.99 | 7 | 9220 | 11.8 | 6 | 9500 | 12.4 | 6 | 9620 | 10.3 | | | | | | |
| Cₘₐₓ | 7 | 76.3 | 9.32 | 7 | 80.3 | 14.6 | 6 | 78.3 | 15.2 | 6 | 84.1 | 11.3 | | | | | | |
| [nM/L] | | | | | | | | | | | | | | | | | | |

DG2 (weekly administration)

| Week | 1 | | | 3 | | | 5 | | | 6 | | | 7 | | | 9 | | |
|---|---|---|---|---|---|---|---|---|---|---|---|---|---|---|---|---|---|---|
| Dose | 0.6 mg | | | 1.2 mg | | | 1.8 mg | | | 1.8 mg | | | 2.4 mg | | | 3.0 mg | | |
| | | | gCV | | | gCV | | | gCV | | | gCV | | | gCV | | | gCV |
| | N | gMean | [%] | N | gMean | [%] | N | gMean | [%] | N | gMean | [%] | N | gMean | [%] | N | gMean | [%] |
| AUC¹ | 8 | 2020 | 18.6 | 7 | 4930 | 21.7 | 3 | 6640 | 25.9 | 4 | 7550 | 18.2 | 4 | 9830 | 14.1 | 3 | 13700 | 15.7 |
| Cₘₐₓ | 9 | 16.9 | 20.5 | 7 | 42.8 | 28.9 | 4 | 57.3 | 23.2 | 4 | 66.9 | 20.9 | 4 | 81.8 | 9.26 | 3 | 120 | 12.5 |

| [nM/L] | | | | | | | | | | | | |
|---|---|---|---|---|---|---|---|---|---|---|---|---|
| DG2 (weekly administration) | | | | | | | | | | | | |
| Week | 11 | | | 13 | | | 15 | | | 16 | | |
| Dose | 3.6 mg | | | 4.2 mg | | | 4.8 mg | | | 4.8 mg | | |
| | | | gCV | | | gCV | | | gCV | | | gCV |
| | N | gMean | [%] | N | gMean | [%] | N | gMean | [%] | N | gMean | [%] |
| AUC¹ | 3 | 16400 | 11.0 | 3 | 19300 | 12.2 | 3 | 22900 | 13.6 | — | — | — |
| Cₘₐₓ | 3 | 139 | 8.67 | 3 | 162 | 10.2 | 3 | 196 | 18.0 | 3 | 201 | 7.77 |
| [nM/L] | | | | | | | | | | | | |

DG3 (twice weekly administration)

| Week | 1 | | | 3 | | | 5 | | | 6 | | | 7 | | | 9 | | |
|---|---|---|---|---|---|---|---|---|---|---|---|---|---|---|---|---|---|---|
| Dose | 0.3 mg | | | 0.6 mg | | | 0.9 mg | | | 0.9 mg | | | 1.2 mg | | | 1.5 mg | | |
| | | | gCV | | | gCV | | | gCV | | | gCV | | | gCV | | | gCV |
| | N | gMean | [%] | N | gMean | [%] | N | g Mean | [%] | N | gMean | [%] | N | gMean | [%] | N | gMean | [%] |
| AUC¹ | — | — | — | 9 | 1640 | 14.8 | 9 | 3520 | 13.9 | 8 | 3830 | 16.6 | 9 | 4690 | 15.1 | 8 | 6720 | 19.3 |
| Cₘₐₓ | 9 | 7.76 | 14.7 | 9 | 27.3 | 14.3 | 9 | 53.7 | 9.68 | 9 | 65.0 | 17.6 | 9 | 76.2 | 12.8 | 9 | 116 | 20.3 |
| [nM/L] | | | | | | | | | | | | | | | | | | |
| DG3 (twice weekly administration) | | | | | | | | | | | | | | | | | | |
| Week | 11 | | | 13 | | | 15 | | | 16 | | | | | | | | |
| Dose | 1.8 mg | | | 2.1 mg | | | 2.4 mg | | | 2.4 mg | | | | | | | | |
| | | | gCV | | | gCV | | | gCV | | | gCV | | | | | | |
| | N | gMean | [%] | N | gMean | [%] | N | gMean | [%] | N | gMean | [%] | | | | | | |
| AUC¹ | 9 | 8210 | 18.1 | 7 | 9780 | 15.5 | 7 | 10200 | 13.8 | 7 | 11200 | 12.7 | | | | | | |
| Cₘₐₓ | 9 | 137 | 16.2 | 9 | 170 | 21.7 | 7 | 175 | 18.1 | 7 | 181 | 12.7 | | | | | | |
| [nM/L] | | | | | | | | | | | | | | | | | | |

¹AUC₀₋₁₆₈ [nM*h]

### Safety Results

The cumulative percentage (%) of subjects withdrawn from the up-titration by the dose escalation scheme, namely the pre-specified primary safety endpoint, was higher in DG2 (66.7%, 6 subjects) than DG1 (22.2%, 2 subjects) and DG3 (22.2%, 2 subjects). Overall, 10 subjects (37.0%) treated with Compound I withdrew from the pre-planned up-titration, while none of the subjects in the placebo group withdrew (Table 10).

TABLE 10
Stop of up-titration overall - TS

| | | Number of subjects withdrawn | |
|---|---|---|---|
| | Number of | from the up-titration | |
| Treatment group | subjects | N | (%) |
| Placebo | 9 | 0 | (0.0) |
| DG1 | 9 | 2 | (22.2) |
| DG2 | 9 | 6 | (66.7) |
| DG3 | 9 | 2 | (22.2) |
| Compound I Total | 27 | 10 | (37.0) |

A total of 35 patients (97.2% of 36 trial subjects) had at least 1 AE during the on-treatment period. Two subjects (5.6% of 36 trial subjects) were reported with AEs of sever intensity: 1 (11.1% of 9 trial subjects) in DG2 and DG3 each. All 27 subjects in the Compound I dosing groups (i.e. DGs 1 to 3) had AEs that were deemed as related to Compound I by the investigator. Two subjects (5.6% of 36 trial subjects), who were both in DG1, had AEs leading to treatment discontinuation. One subject (2.8% of 36 trial subjects) in DG1 was reported with an SAE. One subject (2.8% of 36 trial subjects) in DG1 was reported with an other significant AE according to ICH E3.

There were neither deaths nor AEs of special interest reported. Table 11 provides an overall summary of AEs during the on-treatment period.

TABLE 11
Overall summary of AEs-TS

| | | | Compound I | | | | | | | | | |
|---|---|---|---|---|---|---|---|---|---|---|---|---|
| | Placebo | | DG1 | | DG2 | | DG3 | | Cmp I Total | | Total | |
| | N | % | N | % | N | % | N | % | N | % | N | % |
| Number of subjects | 9 | 100.0 | 9 | 100.0 | 9 | 100.0 | 9 | 100.0 | 27 | 100.0 | 36 | 100.0 |
| Subjects with any AE | 8 | 88.9 | 9 | 100.0 | 9 | 100.0 | 9 | 100.0 | 27 | 100.0 | 35 | 97.2 |
| Subjects with severe AEs | 0 | 0.0 | 0 | 0.0 | 1 | 11.1 | 1 | 11.1 | 2 | 7.4 | 2 | 5.6 |
| Subjects with investigator | 4 | 44.4 | 9 | 100.0 | 9 | 100.0 | 9 | 100.0 | 27 | 100.0 | 31 | 86.1 |
| defined drug-related AEs | | | | | | | | | | | | |
| Subjects with AEs leading | 0 | 0.0 | 2 | 22.2 | 0 | 0.0 | 0 | 0.0 | 2 | 7.4 | 2 | 5.6 |
| to discontin. of trial drug | | | | | | | | | | | | |
| Subjects with AEs of | 0 | 0.0 | 0 | 0.0 | 0 | 0.0 | 0 | 0.0 | 0 | 0.0 | 0 | 0.0 |
| special interest | | | | | | | | | | | | |
| Subjects with serious AEs | 0 | 0.0 | 1 | 11.1 | 0 | 0.0 | 0 | 0.0 | 1 | 3.7 | 1 | 2.8 |
| Requires or Prolongs | 0 | 0.0 | 1 | 11.1 | 0 | 0.0 | 0 | 0.0 | 1 | 3.7 | 1 | 2.8 |
| Hospitalization | | | | | | | | | | | | |

The most common AEs by system organ class (SOC) were ‘gastrointestinal disorders’ with 28 subjects (77.8% of 36 trial subjects). Other types of AEs reported for at least 20% of subjects overall were ‘general disorders and administration site conditions’ with 16 subjects (44.4% of 36 trial subjects) and ‘metabolism and nutrition disorders’ with 25 subjects (69.4% of 36 trial subjects). There were no AEs reported in the cardiac disorder class. The most common AE by preferred term (PT) was ‘decreased appetite’ with 25 subjects (69.4% of 36 trial subjects): 8 subjects (88.9% of 9 trial subjects) in DG1, 9 subjects (100.0% of 9 trial subjects) in DG2, 7 subjects (77.8% of 9 trial subjects) in DG3, and 1 subject (11.1% of 9 trial subjects) in the placebo group. All AEs of ‘decreased appetite’ reported for subjects in the Compound I dosing groups were deemed related to the trial drug by the investigator, and this was the most common drug-related AE with 24 subjects (66.7% of 36 trial subjects).

Adverse events of severe intensity were reported for 2 subjects (5.6% of 36 trial subjects): 1 subject (11.1% of 9 trial subjects) in DG2 reported severe ‘vomiting’, and 1 subject (11.1% of 9 trial subjects) in DG3 reported severe ‘diarrhoea’. Both were reported as non-serious AEs and were determined to be related to the trial drug by the investigator. All other AEs were of mild or moderate intensity.

Two subjects (5.6% of 36 trial subjects), who were both in DG1, discontinued trial medication because of AEs: 1 AE was ‘vomiting’ of moderate intensity, which was the only one SAE reported in this trial; another was ‘amylase increase’ of mild intensity, which was a non-serious AE, and this non-serious AE leading to treatment discontinuation was the only one other significant AE according to ICH E3 reported in this trial. Both AEs were determined to be related to the trial drug by the investigator and resolved by the end of the trial.

Findings of clinical relevance were not observed regarding clinical laboratory values.

An expected increase in pulse rate was observed in the Compound I dosing groups.

No clinically relevant findings were seen in vital signs, the ECG evaluation, local tolerability, or in suicidal risk assessment.

### Conclusions

On the safety aspect, the investigation of the weekly or twice-weekly up-titration schemes of Compound I administered subcutaneously up to 4.8 mg in healthy Japanese male subjects with BMI 23 to 40 kg/m² revealed no unexpected safety or tolerability concerns. The proportion of subjects who withdrew from the up-titration was lower in the twice-weekly dosing scheme (DG3) than in the weekly dosing scheme (DG2). Most subjects in the Compound I dosing groups were reported with ‘decreased appetite’ in the SOC ‘metabolism and nutrition disorders’, followed by ‘dyspepsia’ in the SOC ‘gastrointestinal disorders’. These AEs are in line with the known side effect profile of GLP1 receptor agonists. The twice-weekly dosing scheme (DG3) showed the lowest frequency of drug-related ‘gastrointestinal disorders’ among the dosing schemes. No clinically relevant cardiac disorders were reported. The safety findings in Japanese subjects were similar to the previous Phase I trial conducted overseas.

Following administration of Compound I, exposures in terms of Cₘₐₓ, AUC₀₋₁₆₈ (DG1 and DG2) and AUC₀₋₇₂ (DG3) increased with increasing doses.

### Example 3: A Phase II Dose-Finding Study in Patients with Type 2 Diabetes Mellitus

A Phase II, randomized, parallel group, dose-finding study of subcutaneously administered Compound I for 16 weeks, compared with placebo and open-label semaglutide in patients with type 2 diabetes mellitus

### Objective

The primary objective was to demonstrate proof of clinical concept (PoCC) with respect to a non-flat dose response curve and to define a suitable dose escalation scheme and dose range for Compound I regarding safety, tolerability, and efficacy.

### Methodology

Randomized, placebo and active comparator controlled, double-blind within dose groups, parallel-group, 16-week trial. An open-label arm (semaglutide) was included as benchmark to compare response curves and support assumptions for Phase III design.

### Number of Subjects

Planned: Entered: Approximately 410
Actual: Screened: 669
Entered: 413

| | | | Analyzed(primary |
|---|---|---|---|
| | Entered | Treated | endpoint) |
| Overall | 413 | 411 | 411 |
| Placebo | 60 | 59 | 59 |
| Dose group 1 | 50 | 50 | 50 |
| Dose group 2 | 50 | 50 | 50 |
| Dose group 3 | 52 | 52 | 52 |
| Dose group 4 | 50 | 50 | 50 |
| Dose group 5 | 51 | 51 | 51 |
| Dose group 6 | 50 | 49 | 49 |
| Semaglutide | 50 | 50 | 50 |

### Diagnosis

Patients with type 2 diabetes mellitus with insufficient glycaemic control despite diet, exercise and metformin treatment

### Main Criteria for Inclusion

Patients with type 2 diabetes mellitus for at least 6 months prior to informed consent who had a baseline HbA1c 7.0%-10.0% (both inclusive), treatment with a stable dose of metformin ≥1000 mg/day for at least 3 months prior to screening, and body mass index (BMI) 25 kq/m₂-50 kg/m₂ (both inclusive) at screening. Investigational product: Compound I

### Dose:

Dose group 1: 0.3 mg once weekly
Dose group 2: 0.3, 0.3, 0.6, 0.6, 0.9 mg (weeks 5−16) once weekly
Dose group 3: 0.3, 0.6, 0.9, 1.2, 1.5, 1.8 mg (weeks 6−16) once weekly
Dose group 4: 0.6, 0.6, 1.2, 1.2, 1.8, 2.4, 2.7 mg (weeks 7−16) once weekly
Dose group 5: 0.3, 0.3, 0.6, 0.6, 0.9, 0.9, 1.2 mg (weeks 7−16) twice weekly
Dose group 6: 0.3, 0.6, 0.9, 1.2, 1.5, 1.5, 1.8 mg (weeks 7−16) twice weekly

### Mode of Administration:

Solution for injection

### Investigational Product:

Placebo
Dose: Not applicable
Mode of administration: Solution for injection

### Comparator Product:

Semaglutide
Dose: 0.25, 0.25, 0.25, 0.25, 0.5, 0.5, 0.5, 0.5, 1.0 mg (weeks 9−16) once weekly
Mode of administration: Solution for injection

### Duration of Treatment

16 weeks

### Efficacy and Other Criteria for Evaluation

The primary endpoint was the absolute change in HbA1c from baseline to 16 weeks.

The key secondary endpoint was the relative body weight change from baseline to 16 weeks Secondary endpoints included:
The absolute body weight change from baseline to 16 weeks
The absolute change in waist circumference from baseline to 16 weeks
The percentage of patients with 5% or greater body weight loss from baseline to 16 weeks

The percentage of patients with 10% or greater body weight loss from baseline to 16 weeks

### Safety Criteria for Evaluation

Safety endpoints included Columbia-Suicide Severity Rating Scale (C-SSRS) and various electrocardiograms (ECG) assessments. Safety was assessed based on adverse events (AEs), adverse event of special interest (AESIs), clinical laboratory assessments, vital signs, and physical examination. AESIs were pre-specified in the protocol as pancreatitis and hepatic injury.

### Statistical Methods

Primary endpoint: the analyses for PoCC and dose-finding was performed using a multiple comparison procedure and modelling (MCP-Mod). First, a mixed effect model for repeated measurements (MMRM) was calculated to estimate the treatment effect in each dose group and the corresponding covariance matrix. Using the MCP-Mod approach, these estimates were then further used to (1) test for a non-flat dose response curve and (2) identify suitable dose-response shapes based on a selection of candidate models pre-defined in the protocol. The final model was then calculated by averaging overall significant model shapes.

Secondary endpoints: the key secondary endpoint was analyzed using the same MCP-Mod approach as for the primary endpoint. The absolute body weight change and the change in waist circumference were assessed using MMRM. The percentages of patients with ≥5% or ≥10% body weight loss from baseline to 16 weeks were analyzed using logistic regression and descriptive statistics.

Analyses of safety were descriptive.

No interim analysis was conducted.

### Summary—Conclusions

The 6 Compound I dose groups are named in this section (Example 3) based on the maintenance dose, i.e. dose group 1 is referred to as Compound I 0.3 mg, dose group 2 as Compound I 0.9 mg, dose group 3 as Compound I 1.8 mg, dose group 4 as Compound I 2.7 mg, dose group 5 as Compound I 1.2 biw (2.4) mg, and dose group 6 as Compound I 1.8 biw (3.6) mg.

Trial Subjects and Compliance with the Clinical Trial Protocol

Of the 413 randomized patients, 398 patients (96.8%) completed the trial, which included patients who prematurely discontinued treatment but completed the observational period as planned. Of the 411 patients treated with trial medication, 80 patients (19.5%) prematurely discontinued treatment. The most common reason for premature discontinuation from trial medication was an AE (53 patients, 12.9% overall), with higher frequency reported for the Compound I groups than for the placebo group and the semaglutide group.

The main patient characteristics are summarised in Table 12. Demographic data, baseline characteristics, concomitant therapies, and medical history were mostly balanced across treatment groups; some imbalances between groups were noted. A total of 62 randomized patients (15%) had at least 1 important protocol deviation leading to exclusion from the per protocol set. The most common category was “prohibited medication use” (41 patients /9.9%). Median treatment duration (weeks) was 15.14 (Q1, Q3 15.00, 15.14) for Compound I 0.3 mg, 15.14 (15.14, 15.14) for Compound I 0.9 mg, 15.14 (8.57, 15.14) for Compound I 1.8 mg, 15.14 (3.86, 15.14) for Compound 12.7 mg, 15.57 (15.57, 15.71) for Compound I 1.2 biw (2.4) mg, 15.57 (14.86, 15.71) for Compound I 1.8 biw (3.6) mg, 15.14 (15.00, 15.57) for placebo, and 15.14 (15.14, 15.14) for semaglutide.

TABLE 12
Selected demographic data and selected baseline characteristics - Treated set

| Characteristics at baseline | Total | Characteristics at baseline (cont.) | Total |
|---|---|---|---|
| Number of patients, N (%) | 411 (100.0) | BMI [kg/m²], mean (SD) | 33.86 (6.00) |
| Gender, N (%) | | <30, N (%) | 118 (28.7) |
| Male | 233 (56.7) | 30 to <35, N (%) | 134 (32.6) |
| Female | 178 (43.3) | ≥35, N (%) | 159 (38.7) |
| Region, N (%) | | Time from 1^{st} diagnosis [years], | 7.59 (5.79) |
| North America | 192 (46.7) | mean (SD) | |
| Europe | 153 (37.2) | <1, N (%) | 21 (5.1) |
| Asia-Pacific | 56 (16.1) | 1 to <5, N (%) | 142 (34.5) |
| Race, N (%) | | 5 to <10, N (%) | 132 (32.1) |
| White | 344 (83.7) | ≥10, N (%) | 116 (28.2) |
| Asian | 42 (10.2) | HbA1c [%], mean (SD) | 8.07 (0.84) |
| Black/African American | 20 (4.9) | <7.0 | 20 (4.9) |
| Ethnicity, N (%) | | 7.0 to <8.0 | 193 (47.0) |
| Not Hispanic/Latino | 320 (77.9) | 8.0 to <9.0 | 130 (31.6) |
| Hispanic/Latino | 91 (22.1) | 9.0 to <10.0 | 60 (14.6) |
| Age [years], mean (SD) | 57.3 (9.8) | ≥10 | 8 (1.9) |
| <65, N (%) | 309 (75.2) | Weight [kg], mean (SD) | 96.55 (21.54) |
| ≥65, N (%) | 102 (24.8) | <70 | 39 (9.5) |
| Waist circumference [cm], mean | 110.3 (18.2) | 70 to <80 | 53 (12.9) |
| (SD) | | 80 to <90 | 77 (18.7) |
| | | ≥90 | 242 (58.9) |

BMI = body mass index;
SD = standard deviation

### Efficacy

### Primary Endpoint

HbA1c level decreased from baseline until 16 weeks of treatment in all Compound I dose groups. The decrease from baseline was significantly greater for all Compound I dose groups compared with placebo at all tested time points. Maximum absolute decrease from baseline in HbA1c (%) was observed in the Compound I 1.8 mg dose group at Week 17 (adjusted mean MMRM estimate=−1.72, 95% Cl −1.94, −1.49), with similar results observed in other Compound I dose groups, except for the lowest dose of 0.3 mg.

Based on the MCP-Mod, the predicted dose-response curve of absolute change from baseline in HbA1c after 16 weeks of treatment reached plateau at the Compound I 1.8 mg once weekly dose.

In the descriptive analysis of the primary endpoint, the 4 higher Compound I dose groups compared favorably to semaglutide (mean absolute decrease in HbA1c (%) from baseline at Week 17=−1.79 (SD 0.92) for Compound I 1.8 mg, −1.67 (0.78) for Compound I 2.7 mg, −1.68 (0.90) for Compound I 1.2 biw (2.4) mg, −1.79 (0.76) for Compound I 1.8 biw (3.6) mg, and −1.50 (0.84) for semaglutide).

### Key Secondary Endpoint

Relative body weight loss from baseline during 16 weeks of treatment was observed in all Compound I dose groups in a clear dose-dependent manner. The decrease from baseline was significantly greater for all Compound I dose groups compared with placebo at Week 17, except for the lowest dose of 0.3 mg. Maximum relative body weight loss (%) from baseline was detected in the Compound I 1.8 biw (3.6) mg dose group at Week 17 (adjusted mean MMRM estimate=−8.68, 95% Cl −10.06, −7.30).

Based on the MCP-Mod, the predicted dose response of relative body weight loss from baseline after 16 weeks of treatment did not reach plateau with the Compound I doses investigated in this trial. A clear dose-dependent effect was shown with maximum effect on weight loss predicted for the Compound I 1.8 biw (3.6) mg dose.

In the descriptive analysis of the key secondary endpoint, the 4 higher Compound I dose groups compared favorably to semaglutide (mean relative body weight loss (%) from baseline at Week 17=−6.63 (SD 5.13) for Compound I 1.8 mg, −6.68 (4.05) for Compound I 2.7 mg, −7.16 (6.06) for Compound I 1.2 biw (2.4) mg, −8.95 (5.33) for Compound I 1.8 biw (3.6) mg, and −5.40 (4.33) for semaglutide).

### Secondary Endpoints

Absolute Body Weight Change from Baseline to 16 Weeks

Maximum absolute body weight loss (kg) from baseline was detected in the Compound I 1.8 biw (3.6) mg dose group at Week 17 (adjusted mean=−8.38, 95% Cl −9.68, −7.08). At Week 17, the decrease from baseline in absolute body weight loss was significantly greater than placebo for all Compound I dose groups, except for the lowest dose of 0.3 mg. The 4 higher Compound I dose groups (1.8 mg, 2.7 mg, 1.2 biw (2.4) mg, and 1.8 biw (3.6) mg) compared favorably to semaglutide in terms of body weight loss from baseline.

Absolute Change in Waist Circumference from Baseline to 16 Weeks

Maximum decrease in absolute waist circumference (cm) from baseline was detected in the Compound I 1.8 biw (3.6) mg dose group at Week 17 (adjusted mean MMRM estimate=−10.49, 95% Cl −13.84, −7.14), compared favorably to semaglutide (−4.82, 95% Cl −7.79, −1.84). Data for the absolute change from baseline in waist circumference were highly variable with wide confidence intervals.

Percentage of patients with ≥5% body weight loss from baseline to 16 weeks The proportion of patients with ≥5% body weight loss from baseline after 16 weeks of treatment increased with increasing Compound I doses. More than 50% of patients treated with Compound I 1.2 biw (2.4) mg or Compound I 1.8 biw (3.6) mg attained a ≥5% body weight loss. In the semaglutide group, 38.0% of patients had ≥5% body weight loss.

Percentage of patients with ≥10% body weight loss from baseline to 16 weeks The proportion of patients with ≥10% body weight loss from baseline after 16 weeks of treatment increased dose-dependently. Approximately one-fourth of patients treated with Compound I 1.2 biw (2.4) mg and one-third of patients treated with Compound I 1.8 biw (3.6) mg could attain a 210% body weight loss. In the semaglutide group, 16.0% of patients had 210% body weight loss.

### Safety Results

### Adverse Events

Compared with the placebo group and the semaglutide group, all Compound I groups had higher frequencies of patients with at least one reported AE, investigator-defined drug-related AEs, and AEs leading to discontinuation of trial medication. Severe AEs and serious AEs were reported at low frequencies in the Compound I groups and the placebo group; there were no severe AEs or serious AEs reported in the semaglutide group. All serious AEs were in the category “required or prolonged hospitalisation” (Table 13).

TABLE 13
Overall summary of adverse events-TS

| Category of AEs | Placebo | Cmp I | Cmp I | Cmp I | Cmp I | Cmp I | Cmp I | Sema- | |
|---|---|---|---|---|---|---|---|---|---|
| | | Total N (%) | Cmp I | 0.3 mg | 0.9 mg | 1.8 mg | 2.7 mg | 1.2 biw | 1.8 |
| | | glutide | | | | | | | |
| | biw | N (%) | N (%) | N (%) | N (%) | N (%) | N (%) | (2.4) mg | (3.6) mg |
| | | | | | | | | N (%) | N (%) |
| Number of patients | 59 | 50 | 50 | 52 | 50 | 51 | 49 | 50 | 302 |
| | (100.0) | (100.0) | (100.0) | (100.0) | (100.0) | (100.0) | (100.0) | (100.0) | (100.0) |
| Any AE | 31 | 33 | 38 | 42 | 41 | 39 | 42 | 26 | 235 |
| | (52.5) | (66.0) | (76.0) | (80.8) | (82.0) | (76.5) | (85.7) | (52.0) | (77.8) |
| Severe AEs | 4 (6.8) | 3 (6.0) | 1 (2.0) | 4 (7.7) | 3 (6.0) | 2 (3.9) | 3 (6.1) | 0 | 16 (5.3) |
| Investigator | 13 | 25 | 26 | 33 | 29 | 28 | 36 | 19 | 177 |
| defined drug- | (22.0) | (50.0) | (52.0) | (63.5) | (58.0) | (54.9) | (73.5) | (38.0) | (58.6) |
| related AEs | | | | | | | | | |
| AEs leading to | 3 | 5 | 5 | 11 | 15 | 4 | 8 | 2 | 48 |
| discontinuation | (5.1) | (10.0) | (10.0) | (21.2) | (30.0) | (7.8) | (16.3) | (4.0) | (15.9) |
| of trial | | | | | | | | | |
| medication | | | | | | | | | |
| AEs of special | 0 | 0 | 0 | 1 (1.9) | 0 | 0 | 0 | 0 | 1 |
| (0.3) interest | | | | | | | | | |
| Serious AEs | 3 (5.1) | 1 (2.0) | 4 (8.0) | 3 (5.8) | 2 (4.0) | 1 (2.0) | 0 | 0 | 11 (3.6) |

Adverse events were most frequently reported in the SOC gastrointestinal disorders, followed by metabolism and nutrition disorders, and reported more frequently for the Compound I groups than for the placebo group and the semaglutide group. On the PT level, the most frequently reported AEs were nausea (all Compound I dose groups: 109 patients/36.1%, placebo: 5 patients/8.5%, semaglutide: 6 patients/12.0%), vomiting (all Compound I dose groups: 58 patients/19.2%, placebo: 3 patients/5.1%, semaglutide: 2 patients/4.0%), and diarrhea (all Compound I dose groups: 56 patients/18.5%, placebo: 7 patients/11.9%, semaglutide: 5 patients/10.0%). SAEs were reported for 11 patients (3.6%) in all Compound I dose groups, 3 patients (5.1%) in the placebo group, and 0 patients in the semaglutide group. Serious gastrointestinal AEs were reported for 4 patients (1.3%) in all Compound I dose groups. SAEs assessed as drug-related by the investigator were reported for 4 patients (1.3%) in all Compound I dose groups and 0 patients in either placebo group or semaglutide group. Serious gastrointestinal AEs assessed as drug-related by the investigator were reported for 3 patients (1.0%) in all Compound I dose groups. One AESI of hepatic injury was reported. There were no cases of pancreatitis. There were no deaths in the trial.

### Laboratory Parameters

There were few patients with possibly clinically significant abnormalities (PCSA) in clinical laboratory parameters. There was no Compound I dose-dependency in terms of PCSA. Exceptions were proportion of patients with PCSA high glucose, which decreased in the following groups: Compound I 0.9 mg (26.0% at baseline, 13.9% on treatment), Compound I 1.8 mg (34.6% at baseline, 9.1% on treatment), Compound I 2.7 mg (22.0% at baseline, 5.4% on treatment), Compound I 1.2 biw (2.4) mg (29.4% at baseline, 0.0% on treatment), Compound I 1.8 biw (3.6) mg (24.5% at baseline, 5.6% on treatment), and semaglutide (20.0% at baseline, 5.0% on treatment). The proportion of patients with PCSA high glucose in the placebo group and the Compound I 0.3 mg dose group did not decrease during treatment.

There were no relevant changes in the other safety laboratory parameters.

### Vital Signs, Including ECG Safety Endpoints

ECG notable findings at any time on treatment were reported for a few patients. There were no obvious differences in terms of ECG notable findings between the Compound I groups, the placebo group, and the semaglutide group.

Maximum QT(c) changes from baseline on treatment and new onsets in terms of QT(c) interval and morphological findings were balanced across treatment groups.

New onsets on treatment by overall ECG interpretation and clinical relevance of findings were reported for 6 patients (10.2%) in the placebo group, 11 patients (22.0%) in the Compound I 0.3 mg group, 10 patients (20.4%) in the Compound I 0.9 mg group, 11 patients (22.0%) in the Compound I 1.8 mg group, 9 patients (18.8%) in the Compound I 2.7 mg group, 12 patients (24.0%) in the Compound I 1.2 biw (2.4) mg group, 11 patients (22.9%) in the Compound I 1.8 biw (3.6) mg group, and 14 patients (28.0%) in the semaglutide group.

Heart rate increase from baseline was observed in the 4 higher Compound I dose groups (1.8 mg, 2.7 mg, 1.2 biw (2.4) mg, and 1.8 biw (3.6) mg) and the semaglutide group. The most pronounced increase from baseline in heart rate was observed in the Compound I 2.7 mg group. Mean increases from baseline in heart rate were below 10 beats/min at all time points, except for 2 time points in the Compound I 2.7 mg group (10.32 beats/min at Week 7 and 10.06 beats/min at Week 16).

There were no relevant changes in other vital sign parameters.

### Columbia Suicide Severity Rating Scale (C-SSRS)

One patient in the placebo group reported thoughts of suicide that were already present at Screening and continued until Week 3 in the trial.

### NASH Scores

Mean fibrosis-4, AST to platelet ratio index (APRI), and non-alcoholic fatty liver disease (NAFLD) fibrosis scores at baseline were comparable between treatment groups.

Changes from baseline in fibrosis-4 scores were minor in all treatment groups. There were no incidences of liver fibrosis or advanced liver damage.

### CONCLUSIONS

Compound I significantly reduced HbA1c and body weight in patients with T2DM and obesity/overweight. At doses of 1.8 mg, 2.7 mg, 1.2 biw (2.4) mg, and 1.8 biw (3.6) mg, Compound I compared favorably to semaglutide in terms of HbA1c level and body weight loss, indicating advantages of Compound I as a dual GLP-1R and GCGR agonist over semaglutide. Compound I showed an overall good safety profile during the trial with gastrointestinal events (nausea, vomiting, and diarrhea) being the most common AEs. No new or unexpected safety or tolerability concerns were raised. The results of this trial suggest further investigation to explore clinical effect and optimize treatment regimen of Compound I in the indications of T2DM, chronic weight management, and NASH.

1. A method for administering a peptidic GLP1/glucagon receptor agonist comprising administering at least two consecutive administrations of an effective amount of the agonist to a human in need thereof, wherein the agonist has a plasma half-life in humans of at least 60 hours, wherein the interval between any two consecutive subcutaneous administrations of the agonist is such that the ratio between the plasma half-life in humans of the agonist (in hours) and the administration interval (in hours) is more than 1.0.

2. The method according to claim 1, wherein the ratio between the plasma half-life in humans of the agonist and the administration interval is between 1.0 and 5.0.

3. The method according to claim 1, wherein the agonist is administered sub-chronically or chronically).

4. The method according to claim 1, wherein the agonist is a glucagon analogue with a sequence identity of 50% or higher when compared to human glucagon.

5. The method according to claim 1, wherein the agonist is
H-H-Ac4c-QGTFTSDYSKYLDERAAKDFI-K([17-carboxy-heptadecanoyl]-isoGlu-GSGSGG)-WLESA-NH₂ (Compound I) (SEQ ID NO:1).

6. The method according to claim 5, wherein the interval between the two consecutive injections of Compound I is shorter than 100 h.

7. The method according to claim 6, wherein the interval between the two consecutive injections of Compound I is shorter than 96 h.

8. The method according to claim 1, wherein the method is used during a dose escalation of the agonist.

9. The method of claim 1, wherein
the interval between any two consecutive administrations is such that the ratio between the plasma half-life in humans of the agonist and the administration interval is between 1.0 and 5.0.

10. The method according to claim 9, wherein the interval between any two consecutive administrations is such that the ratio between the plasma half-life in humans of the agonist and the administration interval is between 1.5 and 5.0.

11. The method according to claim 9, wherein the interval between any two consecutive administrations is such that the ratio between the plasma half-life in humans of the agonist and the administration interval is between 2.0 and 5.0.

12. The method according to claim 7, wherein the interval between two consecutive injections is shorter than 84 hours.

13. (canceled)

14. The method of claim 1, wherein the method is effective for treating a disease or condition comprising type 2 diabetes mellitus (T2DM), obesity or NASH in a human.

15. A method for treating a disease or condition, comprising administering at least two consecutive administrations of an effective amount of a pharmaceutical composition via subcutaneous injection, wherein the pharmaceutical composition comprises a peptidic GLP1/Glucagon receptor agonist, wherein
a) the agonist has a plasma half-life in humans of at least 60 hours;
b) the composition is subcutaneously administered at least two times;
c) the interval between any two consecutive administrations of the agonist is such that the ratio between the plasma half-life in humans of the agonist (in hours) and the administration interval (in hours) is more than 1.02 and wherein the disease or condition is Type 2 diabetes mellitus, obesity, or NASH.

16. The method of claim 15, wherein the agonist is Compound I (SEQ ID NO:1).
